(19) United States
(12) Patent Application Publication
(10) Pub. No.: US 2026/0348970 A1
(43) Pub. Date: Nov. 5, 2026

(54) HIGH-THROUGHPUT IDENTIFICATION METHOD FOR ANTIBODY EPITOPE BASED ON POLYPEPTIDE CHIP

(71) Applicant: ICARBONX (ZHUHAI) COMPANY LIMITED, Zhuhai (CN)

(72) Inventors: Jun Wang, Zhuhai (CN); Yingrui Li, Zhuhai (CN); Binghang Liu, Zhuhai (CN); Chunlan Xie, Zhuhai (CN); Xingwen Ji, Zhuhai (CN); Danni Li, Zhuhai (CN); Bangzhu Xiong, Zhuhai (CN)

(21) Appl. No.: 19/108,145

(22) PCT Filed: Aug. 24, 2023
(86) PCT No.: PCT/CN2023/114686

§ 371 Date: Feb. 28, 2025

(30) Foreign Application Priority Data
Sep. 1, 2022 (CN) 202211067234.9

(51) Int. Cl. G01N 33/68; G01N 33/543

(52) U.S. Cl. CPC G01N 33/6845; G01N 33/543; G01N 33/6857

(57) ABSTRACT

The present disclosure relates to a method for identifying an antigen epitope. The method comprises: acquiring, in a polypeptide chip, a set of amino acid identifier sequences that bind to an antibody to be tested so as to generate a signal, and on the basis of the set of amino acid identifier sequences, generating a set of key amino acid identifier sequences; on the basis of the position, in the space structure of a targeted protein, of a short peptide that binds to said antibody so as to generate a strong signal in the polypeptide chip, generating a spatial alignment region; and on the basis of the spatial alignment region, identifying key binding sites of an antigen epitope contained in the spatial alignment region. According to the method, on the basis that the binding sites of an antigen spatial epitope are obtained by means of identification, the key binding sites exerting a key binding function in the antigen spatial epitope can further be selected.

### TECHNICAL FIELD

The present disclosure belongs to the field of biotechnology, and particularly relates to a high-throughput identification method for antibody epitopes based on polypeptide chip.

### BACKGROUND

An epitope is a specific chemical group present in an antigen molecule that determines the specificity of the antigen. In protein antigens, due to the relative complexity of their structure, they often contain a variety of different epitopes. The epitopes composed of short peptides with continuous linear arrangement of amino acid residues are linear epitopes; although some amino acids are not continuously arranged linearly, they form specific conformations in space, which are called spatial epitopes. Epitopes are the basis of protein antigenicity, so it is of great significance to study protein epitopes for diagnosis and prognosis of diseases, targeted modification of protein molecules to reduce immunogenicity of protein drugs, designing artificial vaccines with no toxic and side effects and immune intervention therapies, etc.

Currently, there are generally three technical solutions for identifying antibody epitopes:

(1) Antigen-antibody crystallization and structure analysis. The antigen protein is synthesized first, which usually by inserting of the protein DNA sequence into a plasmid vector before transfected into E. coli or other active cells for expression in this step. After expression, centrifugation, chromatography and column separation were performed. The heavy and light chains of antibodies are then synthesized using the same approach. Next, the two were mixed and incubated at a molar ratio of 1:1, and the antigen-antibody binding complex was isolated by space exclusion chromatography for crystallization. Diffraction spatial structures (contributing 90% of the structures in the PDB database) were then obtained by X-ray irradiation. The subsequent development of single particle cryoelectron microscopy (Cryo-EM) method, which first freezes the antigen-antibody complexes in glassy ice and then images them with electron microscopy, has become the main tool of structural biology because crystallization is not required. After obtaining the spatial structure, COCOMAP, PYMOL, PISA and other software can be used to predict the bonding and interaction between amino acids based on distances and physical and chemical properties of amino acids, and then determine the main amino acids bound to the epitope region of the antibody. However, the technique of single particle cryoelectron microscope is heavy and complicated, and of high requirements for experimental operators, leading to low throughput and poor stability.

(2) Display system. Both phage display systems and bacterial display systems have been used for attemptation to identify epitopes of antibodies. One idea is to fragment the DNA sequence of antigen protein into random fragments after synthesization and PCR amplification, and then link the fragments to display vectors. The display vector may be a bacterial vector such as a staphylococcal display vector, pSCEM1 or a phage such as pHORF, etc. If transfected into bacterial vector, antibodies are added into cell culture medium, and cells bound with antibody are separated by flow cytometry and further sequenced, and sequencing data are linearly aligned with antigen sequence to locate epitope region. When transfected into phage expression system, antigen protein was displayed on phage surface as fusion protein along with expression of antigen protein and capsid protein. A library is then combined with the antibody coated in ELISA, and the phage that can bind the antibody is isolated and then amplified though infecting host bacteria (such as E. coli ER2738), then the next round of antibody binding-elution-amplification is performed. After 3-5 rounds of this process, a high concentration of phage that can bind to the antibodies can be obtained. The enriched phage was then sequenced to obtain epitope sequences. Another idea is to integrate a large number of random sequences into the expression system. For example, Ph.D-12 phage display system, in which DNA sequences encoding random peptides with 12 amino acids are integrated into the capsid protein of M13 phage, the library theoretically contains about 109 transformation sequences. VirScan integrated DNA sequences encoding 56 amino acids into the T7 bacteriophage display system, both of which were used for attemptation to identify linear and spatial epitopes. The library theoretically contains 108 sequences. These display systems have been developed in three main directions in the antibody field: 1) challenging spatial epitopes, 2) improving throughput, and 3) studying human population virome. The third direction generally assumes that linear epitopes are identified, and therefore directly searches for which peptides appear in which virus protein sequences. The first direction challenges whether the program can identify spatial epitopes. Johan Rockberg demonstrated for the first time in Nature Method in 2008 that bacterial display systems can identify linear epitopes of monoclonal and polyclonal antibodies, and published in Scientific report in 2012 that linear and spatial epitopes can be identified. Another team published an article in 2017 demonstrating that structural epitopes can be identified by forming motifs from signal peptides obtained by expression systems and then spatially aligning motifs to antigenic proteins. The second direction is to challenge whether random sequence library construction can identify epitopes with high throughput. Considering that all the antibodies studied were elicited by short peptides, 112 out of the 174 antibodies studied had signal peptides, and the epitopes of 49/112 of the antibodies could be localized to the eliciting linear peptides. The cutoff of signal peptide is a very sensitive parameter in the research. When cutoff is 3x, 66 antibodies contain 1-20 binding peptides. Considering the relatively small number of peptides, their commonality are mainly studied by observing whether they form significant motifs. The main problems existing in this technical solution are as follows: 1) only peptide signals strongly bound to antibodies will be enriched, which leads to a small number of finalized signal peptides, and weak binding signals cannot be detected; 2) the gap between theoretical peptide space and actual peptide space is unclear, which will affect the stability of detection and the unpredictability of the detection space.

(3) Short peptide chips. A short peptide chip has clear peptide space, and the experimental procedure is simple and stable. And there are many studies show that short peptide chips can be used to identify linear epitopes. There are two existing ideas for identifying epitopes based on short peptides on the chip. One is to linearly align short peptides to antigen sequences, and then enrich and evaluate the aligned regions for scoring. The regions that are significantly enriched and have the highest score are the epitope regions. The second is to find the commonality of short peptides, form a motif, and then align the motif to the linear protein sequence to obtain the epitope region. However, it is not clear whether this method can identify spatial epitopes, how to identify accurate spatial epitopes and to what extent spatial epitopes can be identified.

However, existing mature technologies are often expensive and low throughput, and the potential high throughput solutions have not been proven and are therefore immature. It is difficult to identify spatial epitopes in the prior art, and there are some redundant or unnecessary binding sites in known epitopes. The solution provided by the present disclosure aims at solving the problem whether various types of epitopes can be identified by using polypeptide chips, especially the problem of identifying spatial epitopes, and at the same time, further identifying key binding sites in epitopes which play a crucial role in antigen-antibody binding on the basis of identifying and obtaining spatial epitope binding sites.

### SUMMARY

To overcome the deficiencies of the prior art, the present disclosure provides method for generating binding amino acid identifier sequences based on polypeptide chips to identify spatial epitopes and major binding sites of antigens, intended for use in identifying spatial epitopes of antibody-binding antigens and screening for major binding sites on antigen epitopes.

In one aspect, the present disclosure provides a method for identifying an antigenic epitope, comprising: acquiring a set of amino acid identifier sequences that bind to an antibody to be detected and generate signals in a polypeptide chip, and generating a set of key amino acid identifier sequences based on the set of amino acid identifier sequences;
generating a spatially alignment region based on the position of a short peptide that binds to the antibody to be detected and generates a strong signal in the polypeptide chip in the spatial structure of targeted protein;
identifying key binding sites of the antigenic epitope comprised in the spatially alignment region based on the spatially alignment region.

In another aspect, the present disclosure provides a method for generating an amino acid identifier sequence with a specific length, comprising:
for each amino acid identifier sequence in the set of amino acid identifier sequences, performing the following segmentation operation and generating a set of first subsequences: segmenting the amino acid identifier sequence into first subsequences comprising preset numbers of amino acid identifiers, wherein the amino acid identifiers in the first subsequence appear continuously in the amino acid identifier sequence;
for each amino acid identifier sequence in the set of amino acid identifier sequences, performing a space addition operation and generating a set of second subsequences, wherein the second subsequences are amino acid identifier subsequences comprising preset numbers of amino acid identifiers and the amino acid identifiers in the second subsequence appear non-continuously in the amino acid identifier sequence;
adding the set of first subsequences and the set of second subsequences into the set of amino acid identifier subsequences to generate the set of amino acid identifier subsequences;
wherein performing a space addition and generating a set of second subsequences comprises:
setting that the first and last positions of each amino acid identifier sequence in the set of amino acid identifier sequences are not spaced, determining whether the number of remaining amino acid identifiers in the second subsequence generated after space addition is greater than or equal to a preset space addition number, wherein the number of remaining amino acid identifiers refers to exclusion of the amino acid identifiers at the first and last positions;
if yes, performing a space addition operation on the full amino acid identifier subsequence composed of amino acid identifiers, wherein the space addition operation comprises:
replacing amino acid identifiers in the full amino acid identifier subsequence one by one using from 1 to n designated placeholders to generate a second subsequence with n spaces added;
where n is a preset number of spaces added;
if no, performing an amino acid identifier addition operation on an all-empty subsequence formed by the designated placeholder, wherein the amino acid identifier addition operation comprises:
replacing designated placeholders one by one using from 1 to L−2−n amino acid identifiers to generate a second subsequence comprising L−2−n remaining amino acid identifiers;
wherein L is a preset length of the second subsequence, and n is a preset number of spaces added.

### BRIEF DESCRIPTION OF THE DRAWINGS

The accompanying drawings, which are incorporated herein and constitute as a part of this description, illustrate examples consistent with the description, and serve to explain the principles of the description together with the present description.

FIG. 1 shows the binding map of four antibodies Campath-1H, 9E10, 6A7 and Nivolumab, wherein the abscissa is the Zscore values of antibodies binding to polypeptide chip, the ordinate is the second signal intensity values of antibodies binding to polypeptide chip, the color of each point in the map represents the linear alignment results of key amino acid identifier sequences and targeted antigen proteins, the black ones are the key amino acid identifier sequences that can be linearly aligned with the antigen protein sequences, the gray ones are the key amino acid identifier sequences that cannot be linearly aligned with the antigen protein sequences, the 4aa key amino acid identifier sequences with Zscore values and second signal intensity values both rather large were all obtained after the four antibodies bind with the polypeptide chip and could be linearly aligned with the antigen protein.

FIG. 2 shows the binding map of six antibodies a.4.6.1, F11.2.32, e111, Golimumab, Canakinumab and Bevacizumab, wherein the abscissa is the Zscore values of antibodies binding to polypeptide chip, the ordinate is the second signal intensity values of antibodies binding to polypeptide chip, the color of each point in the map represents the linear alignment results of key amino acid identifier sequences and targeted antigen proteins, the black ones are the key amino acid identifier sequences that can be linearly aligned with the antigen protein sequences, the gray ones are the key amino acid identifier sequences that cannot be linearly aligned with the antigen protein sequences. After binding these six antibodies with the polypeptide chip, only the 4aa key amino acid identifier sequences with Zscore values and second signal intensity values both rather large were obtained, but the 4aa key amino acid identifier sequences that can be linearly aligned with the antigen protein was not obtained.

FIG. 3 shows the binding map of seven poorly binding antibodies S309, hu5c8-Ruplizumab, rhPM-1-Tocilizumab, hu1124-Falizumab, Certolizumab, D2E7-Adalimumab and CR3022, wherein the abscissa is the Zscore values of antibodies binding to polypeptide chip, the ordinate is the second signal intensity values of antibodies binding to polypeptide chip, the color of each point in the map represents the linear alignment results of key amino acid identifier sequences and targeted antigen proteins, the black ones are the key amino acid identifier sequences that can be linearly aligned with the antigen protein sequences, the gray ones are the key amino acid identifier sequences that cannot be linearly aligned with the antigen protein sequences, all of the seven antibodies do not have the 4aa key amino acid identifier sequences with both Zscore values and second signal intensity values both rather large, and the number of the 4aa key amino acid identifier sequences is very small.

FIG. 4 shows the coverage of known sites by sites identified by the present method and sites supported by key amino acid identifier sequences.

FIG. 5 shows the spatial structure of Bax protein predicted by alphafold2. The amino acid exposure area at the positions 13-18 (grey) is large and connected into a ring, while I19 (black) is deep in the depression, exposing very little areas on of the intact protein surface.

FIG. 6 shows the spatial structure (partial) of TNFα protein predicted by alphafold2. The gray+black areas are areas formed by known binding sites, and the black areas are areas formed by identified binding sites.

FIG. 7 shows the relationship between binding intensity information of the binding sites and site scores in spatial epitope identification results of Golimumab antibody. The position shown here is the intact protein position subtracting 76, and the coordinates are consistent with the coordinates of spatially bound free TNFα. Based on the spatially resolved structure, the buried surface area (BSA) and solvation energy effect (DeltaG) of each site can be obtained, wherein the larger BSA is, the greater its contribution to antigen-antibody binding force is, and the smaller DeltaG is, the stronger the binding force may be.

FIG. 8 shows the spatially resolved structure of segment 299-365 of E protein, from spatial structure PDB: 4FFZ. The gray+black ones are known spatial binding sites that essentially encapsulate the structural part. The black ones are three sites where hydrogen bonds and salt bridges form.

FIG. 9 shows the relationship between binding intensity information of the binding sites and site scores in spatial epitope identification results of e11 antibody. Based on the spatially resolved structure, the buried surface area (BSA) and solvation energy effect (DeltaG) of each site can be obtained.

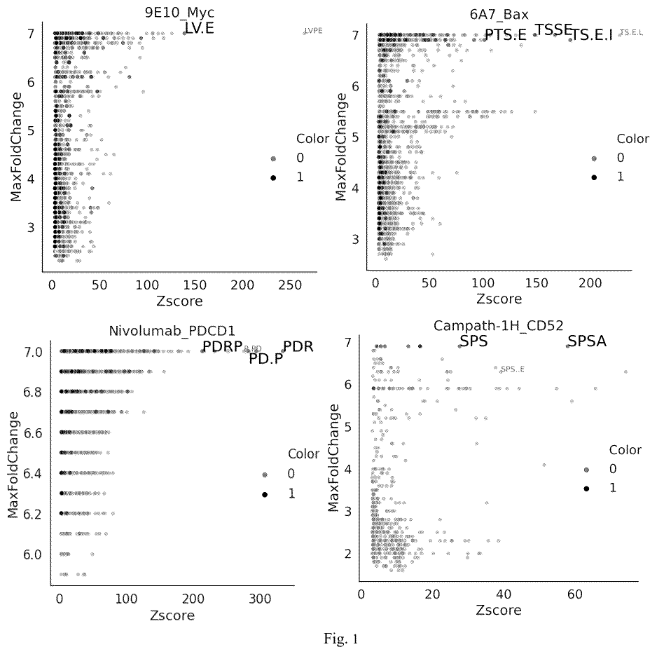

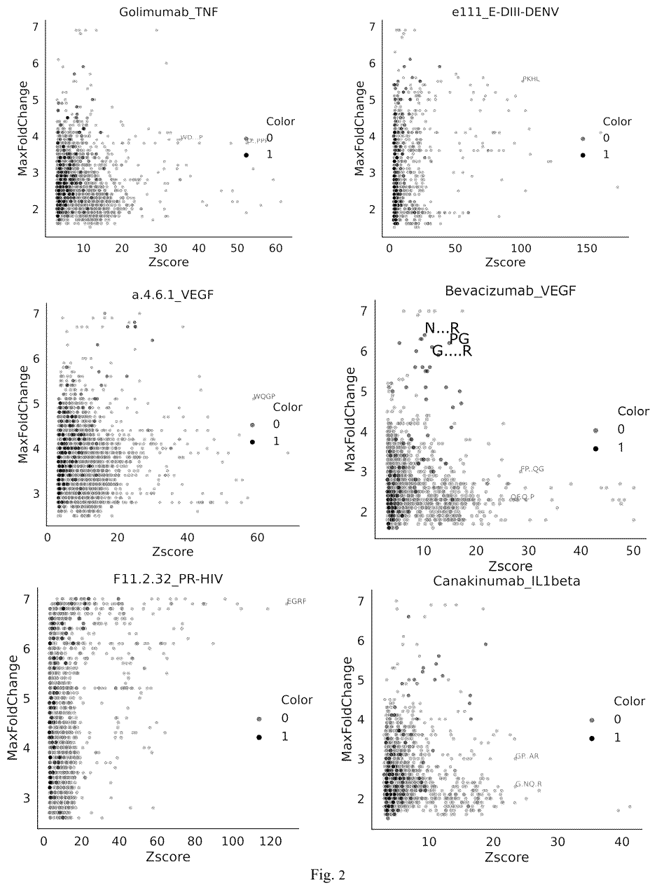

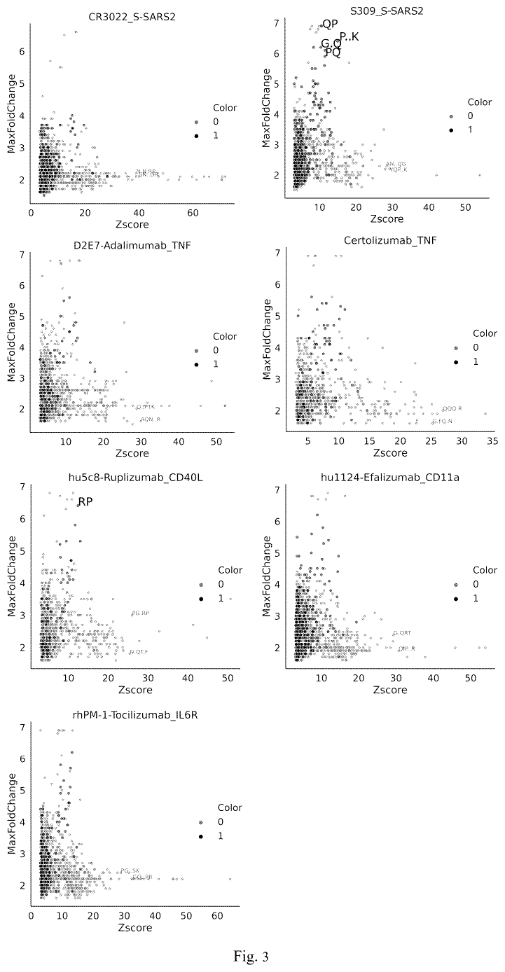

### DETAILED DESCRIPTION OF THE EMBODIMENTS

### I. Definitions

In this disclosure, scientific and technical terms used herein have the meanings commonly understood by those skilled in the art, unless otherwise specified. Also, as used herein, protein and nucleic acid chemistry, molecular biology, cell and tissue culture, microbiology, immunology-related terms and laboratory procedures are terms and general procedures widely used in the respective fields. Meanwhile, for a better understanding of the present disclosure, definitions and explanations of related terms are provided below.

The term “and/or” when used to connect two or more alternatives should be understood to mean any one of the alternatives or any two or more of the alternatives.

As used herein, the term “comprising” or “comprising” means inclusion of a stated element, integer, or step, but not excluding any other element, integer, or step. In this context, when the term “comprises” or “comprising” is used, unless otherwise indicated, it also covers the situation consisting of the stated elements, integers or steps.

As used herein, the term “antibody” is used in its broadest sense and specifically encompasses monoclonal antibodies (including full-length monoclonal antibodies), polyclonal antibodies, multispecific antibodies (e.g., bispecific antibodies), antibody fragments or synthetic polypeptides carrying one or more CDRs or derived from CDR sequences, so long as these polypeptides exhibit the desired biological activity. Antibodies (Abs) and immunoglobulin (Igs) are glycoproteins with the same structural characteristics. “Antibody” may also refer to immunoglobulin and immunoglobulin fragments, whether naturally or partially or wholly synthetically (e.g., recombinantly) produced, including any fragment thereof that retains the binding specificity of a full-length immunoglobulin comprising at least a portion of the variable region of the immunoglobulin molecule. Thus, antibodies include any protein having a binding domain homologous or substantially homologous to an immunoglobulin antigen binding domain (antibody binding site). Antibodies include antibody fragments, such as anti-tumor stem cell antibody fragments. As used herein, the term antibody thus includes synthetic antibodies, recombinantly produced antibodies, multispecific antibodies (e.g., bispecific antibodies), human antibodies, non-human antibodies, humanized antibodies, chimeric antibodies, intracellular antibodies, and antibody fragments such as, but not limited to, Fab fragments, Fab′ fragments, F(ab′)₂ fragments, Fv fragments, disulfide-stabilized Fv (dsFv), Fd fragments, Fd′ fragments, single chain Fv (scFv), single chain Fab (scFab), diabodies, anti-idiotype (anti-Id) antibodies, or antigen binding fragments of any of the above. Antibodies provided herein include members of any immunoglobulin type (e.g., IgG, IgM, IgD, IgE, IgA, and IgY), any class (e.g., IgG1, IgG2, IgG3, IgG4, IgA1, and IgG2), or subclass (e.g., IgG2a and IgG2b) (“type” and “class”, and “subtype” and “subclass” are used interchangeably herein). Natural or wild-type (i.e., derived from non-manipulated members of the population) antibodies and immunoglobulin are typically heterotetrameric glycoproteins of approximately 150,000 daltons, consisting of two identical light chains (L) and two identical heavy chains (H). Each heavy chain has a variable domain (VH) at one end followed by multiple constant domains. Each light chain has a variable domain (VL) at one end and a constant domain at the other end. The “non-manipulated” means that it has not been subjected to treatment intended to cause it to contain or express foreign antigen-binding molecules. Wild-type may refer to the most prevalent allele or species found in a population or to antibodies obtained from non-manipulated animals, as compared to alleles or polymorphisms, or to variants or derivatives obtained by altering amino acids of the antigen-binding molecule by some form of manipulation, e.g., mutagenesis, using recombinant methods, etc.

The term “monoclonal antibody” refers to a population of identical antibodies, meaning that each individual antibody molecule in the monoclonal antibody population is identical to the other antibody molecules. This property is in contrast to that of polyclonal populations of antibodies, which comprise antibodies having a variety of different sequences. Monoclonal antibodies can be prepared by a number of well-known methods, for example, monoclonal antibodies can be prepared by immortalizing B cells, e.g., by fusion with myeloma cells to produce hybridoma cell lines or by infecting B cells with viruses such as EBV. Recombinant techniques can also be used to prepare antibodies from clonal populations of host cells in vitro by transforming the host cells with plasmids carrying artificial sequences of nucleotides encoding the antibodies.

The term “epitope” or “antigenic epitope” includes any determinant capable of binding specifically to an antigen binding protein. Epitope determinants are usually composed of chemically active surface groups of molecules including amino acids or saccharide or glycosyl side chains, such as amino acids, saccharide side chains, phosphoryl or sulfonyl groups, and usually have specific three-dimensional structural characteristics as well as specific charge characteristics. An epitope may be a “linear” or “spatial” structure. In linear epitopes, all interaction sites between a protein and an interacting molecule (e.g., an antibody) are arranged linearly on the primary amino acid sequence of the protein. In spatial epitopes, interaction sites appear on amino acid residues that isolate from each other on proteins.

The term “amino acid identifier sequence” as used herein refers to a sequence of strings derived from a primary amino acid sequence of a protein or peptide chain and representing the primary structure of the protein or peptide chain by specific identifier characters, possibly including both identifier characters representing amino acids and identifier characters representing amino acid positions. Among them, the term “identity” as used herein refers to characters that may represent amino acid positions in amino acids or short peptides, including letters, numbers, operators, punctuation marks, and other symbols, as well as some functional symbols. The term “amino acid identifier” as used herein refers to a character that can represent an amino acid, such as a single letter abbreviation for a common amino acid, or other computer-recognizable characters, such as Greek letters, numbers, etc., can be used to represent an amino acid. For example, the amino acid identifier sequence “RHS” herein contains three amino acid identifiers indicating an amino acid sequence consisting of three amino acids arginine (Arg), histidine (His) and serine (Ser), while the amino acid identifier sequence “R.S” contains two amino acid identifiers and one placeholder (identifier character indicating position of amino acid) indicating an amino acid sequence consisting of three amino acids arginine (Arg), one arbitrary amino acid and serine (Ser).

The term “subsequence” or “substring” as used herein refers to a string of characters derived from the original string, consisting of elements in the original string and/or specified placeholders not contained in the original string. For example, the amino acid identifier subsequence of the amino acid identifier sequence “RHS” includes a subsequence “RHS” composed of three amino acid identifiers and three subsequences “RH”, “HS” and “R.S” composed of two amino acid identifiers, wherein the subsequence “R.S” composed of two amino acid identifiers further includes a designated placeholder.

The term “seed” refers to the starting point of iteration in the growth of a region, and as used herein, seed is a single amino acid site that can be aligned with an amino acid site on a targeted protein. For example, if the spatial structure of a short peptide is aligned with the spatial structure of a target protein, and the result output by the alignment software shows that an amino acid at a certain position in the short peptide matches an amino acid at a corresponding position in the spatial structure of the target protein, then the single amino acid being aligned is the above position, i.e., the seed.

### II. Detailed Description of Specific Embodiments

### 1. Determination of Signal Short Peptides

The signal intensity of monoclonal antibody samples is detected by polypeptide chip. The minimum sample adding unit of the chip is a slide, and each slide is sufficient for repeatedly display of all short peptides in the polypeptide chip. In monoclonal antibody experiments, one set of diluted solutions of the monoclonal antibodies to be tested was added to each slide. In order to evaluate the signal generated by the binding of monoclonal antibodies to short peptides on the chip, space groups were reserved to extract reference signal values. After the signal intensity values of each group was obtained, the larger the first signal intensity value, the stronger the signal, indicating that the binding between the short peptide and the antibody is stronger. Sorting the short peptides in the polypeptide chip in descending order according to the first signal intensity values, and defining the short peptides with the first signal intensity values greater than a preset threshold value and within a preset sorting range as signal short peptides.

The first signal intensity value can be the difference between the logarithmic value of the signal value of the monoclonal antibody group and the logarithmic value of the signal value of the blank group (reference signal intensity value), or it can be the ratio of the signal intensity value of the short peptide divided by the reference signal intensity value.

The reference signal intensity value may be a signal intensity value of a reserved blank group, and if no blank group is reserved, a background signal of the short peptide on the chip that is substantially stable may be used as the reference signal value; or the reference signal value may be set to a user-defined signal intensity value or a signal intensity value of a reference substance or a control sample according to a usage scenario.

After the short peptides in the polypeptide chip are sorted in descending order according to the first signal intensity value, the first 500-2000 short peptides can be selected as signal short peptides according to the total amount of short peptides on the polypeptide chip (for example, the first 500 short peptides, the first 1000 short peptides or the first 2000 short peptides can be selected).

When determining the signal short peptides, for example, (1) when the first signal intensity value is the difference between the logarithmic value of the signal value of the monoclonal antibody group and the logarithmic value of the signal value of the blank group (reference signal intensity value), the first 2000 short peptides with the first signal intensity value>1 are selected as the signal short peptides, indicating that these short peptides on the polypeptide chip can bind with the monoclonal antibody to generate signals; (2) when the first signal intensity value is the ratio of the signal intensity value of the short peptide to the reference signal intensity value, the first 2000 short peptides with the first signal intensity value >2 are taken as the signal short peptides, indicating that these short peptides on the polypeptide chip can bind to the monoclonal antibody to generate signals; when the base number of the logarithmic value obtained in the calculation in (1) is 2, the two cases represented by (1) and (2) are the same.

It is further defined that when the first signal intensity value is the absolute value of the difference between the logarithmic value of the signal value of the monoclonal antibody group and the logarithmic value of the signal value of the blank group (reference signal intensity value), the short peptide with the first signal intensity value >3 is a strong signal short peptide; when the first signal intensity value is the ratio of the signal intensity value of the short peptide divided by the reference signal intensity value, the short peptide with the first signal intensity value >8 is a strong signal short peptide.

### 2. Splitting of Amino Acid Identifier Subsequences

In order to find out how each peptide binds to the antibody, and by which amino acids and combinations of the amino acids, all amino acid identifier subsequences of length <7 (including the first subsequence consisting of all amino acids without space and the second subsequence spaced by the designated placeholder “.”) were generated from the signal peptide. Each subsequence may comprise 1-6 amino acid (aa) identifiers.

For example, all subsequences of an amino acid identifier sequence ABCD include four 1 aa subsequences (A, B, C, D), six 2aa subsequences (AB, A.C, A . . . D, BC, B.D, CD), four 3aa sub-sequences (ABC, AB.D, A.CD, BCD) and one 4aa sub-sequence (ABCD), among which, e.g. “AB”, “ABC” and “BCD” belong to the first subsequence without space, e.g. “A.C”, “A . . . D” and “AB.D” belong to the second subsequence with space.

The algorithm for splitting of subsequences is divided into two parts:

From a given sequence, the first subsequences of all lengths were spilted firet, and then all second subsequences were generated by iterating over space addition, wherein the amino acid positions at the first and last of the subsequence are positions that cannot be added with space. For example, number of the amino acid identifier subsequence of length L (i.e., comprising L amino acid identifiers) can be space added is from 1 to L−2. When the number of spaces is n, determine the relative size of the number of remaining amino acids after spacing L−2−n and the number of spaces n. If L−2−n>n, then the number of remaining amino acid after space addition is more than the number of spaces, and the second subsequence was generated by space adding method; if the number of remaining amino acid after space addition is less than the number of spaces, then the second subsequence was generated by amino acid adding method.

The space adding method refers to a given full amino acid identifier string consisting of amino acids, where the amino acids were replaced by a designated placeholder, such as “.”, to generate second subsequences of various forms with 1 to n spaces added in.

The amino acid adding method refers to a given all-empty string consisting of a designated placeholder, such as “.”, where the “.” were replaced by amino acids given by a specified placeholder, to generate second subsequences of various forms with 1 to L−2−n amino acids added in.

For example, if subsequences of “RHSVV” with two spaces added in were generated, i.e., the length of amino acid identifier subsequence is L=5, the number of spaces is n=2, and the number of spaces is greater than the number of remaining amino acids (L−2−n=1), then the amino acid adding method was used. Finally, subsequences of various forms with one amino acid reserved were generated. For the all-empty string “ . . . ”, there are three possible positions for each one amino acid to be placed in, so three subsequences RH . . . V, R.S.V and R . . . VV were finally generated.

### 3. Definition of Binding Amino Acid Identifier Sequences

(1) Calculate the second signal intensity value of the amino acid identifier subsequences: the binding amino acid identifier sequences correspond to the binding units of the short peptide and the antibody. Firstly, calculate the second signal intensity value of each of the amino acid identifier subsequences generated in the previous step, i.e., the average value of the first signal intensity values of the multiple short peptides comprising the subsequence with the largest first signal intensity value, for example, the average value of the first signal intensity of the short peptide ranked in the top three of the first signal intensity values. The larger the second signal intensity value, the larger the first signal intensity value that can be obtained by the short peptide containing this subsequence, which also means that this subsequence can obtain a stronger signal for the short peptide containing it, and this subsequence is more likely to be a binding unit. However, it is obvious that this index alone cannot define the binding amino acid identifier sequence, because all second signal intensity value a of the subsequence in the short peptides with high first signal intensity values will be relatively large.

(2) Calculation the Zscore of amino acid identifier subsequences: Zscore of each of the amino acid identifier subsequences was further calculated considering the advantages and limitations of the second signal intensity value. Randomly sampling from all short peptides covered by the chip for a preset number of times, and extracting a preset number of short peptides each time; generating all amino acid identifier subsequences of these acquired short peptide, obtain the distribution of the number of short peptides comprising the amino acid identifier subsequence over the preset number of times, and calculate the average value mean and standard deviation sd of the number of times the amino acid identifier subsequence appears;
performing polypeptide chip detection on a sample to be detected to obtain the signal short peptide, generating a set of amino acid identifier subsequences based on the signal short peptide, counting the number of short peptides comprising a certain amino acid identifier subsequence, marked as X;
the number of the extracted preset number of short peptides should be equal to the number of the signal short peptides obtained when the sample to be tested is subjected to the polypeptide chip detection during the above process;

For example, a sample to be tested is subjected to polypeptide chip detection, the first 2000 short peptides with the first signal intensity values >1 are taken as signal short peptides, a set of amino acid identifier subsequences is generated based on the signal short peptides, and the number X of short peptides comprising a certain amino acid identifier subsequence is counted; sample 10000 times randomly from all short peptides covered by the chip, then 2000 short peptides sampled each time, generate all amino acid identifier subsequences of these short peptides and count the number of short peptides comprising the subsequence, and finally obtain the distribution of the number of every subsequence appeared in the 10000 times, calculate the mean value and standard deviation sd, and calculate Zscore according to the following formula:

$$Zscore = \frac{X - mean}{sd}$$

The Zscore value reflects whether the subsequence appears randomly in the signal short peptides. The larger the absolute value of Zscore, the stronger the non-randomness, i.e., the presence or absence of short peptides here is not accidental. When Zscore is large, this subsequence appears more times in the overall signal peptide, theoretically indicating that it will independently bring about the binding of antigen and antibody. However, the subsequence with more amino acids, especially 5aa and 6aa, has low reliability because of its limited coverage of short peptides.

A subsequence with larger Zscore results in binding to the antibody, while a larger value of the second signal intensity means that the subsequence results in stronger binding to the antibody. Therefore, in practice, a Zscore>10 with a second signal intensity value >3 is adopted to define the binding amino acid identifier sequence.

(3) Screening of key amino acid identifier sequences: starting from a 1aa subsequence, recursively filter subsequences with amino acid identifier numbers plus 1. For example, for the subsequence RHS, if the Zscore or the second signal intensity value of the RHS is higher than the Zscore or the second signal intensity value of RH, HS and R.S, then the RHS is considered to be a key amino acid identifier sequence; if the Zscore and the second signal intensity are both less than a subsequence with an amino acid length shorter than that of the RHS, such as RH, then the RHS is considered to have an amino acid S added on the basis of RH, which is not beneficial for binding antibodies to epitopes, thus the RHS is not a key amino acid identifier sequence.

### 4. Strong Signal Short Peptides Alignment to Protein Spatial Structure

Considering the limitation of computational performance, only strong signal short peptides are selected for spatial alignment. The parameter information is “Van del Waals radii sets: Chothia, Probe radius in angstrom:3.9, Calculation mode: Accessible and molecular surface areas”. Then pepSurf was used to align the strong signal short peptides to the spatial structure of the target protein, and the parameter was that the maximum number of iterating cycles should not exceed 100,000. Finally, significant (p<0.05) records of all alignable pairs were obtained. Usually there are 3-5 significant alignment records for each short peptide, so it is necessary to screen the alignment records. In addition to pepurf, spatial structure alignment software such as Pep-3D-Search and PepMapper can also be used.

### 5. Screening of Spatial Comparison Results

After the alignment is completed, there are usually multiple alignment results for each short peptide. Alignment records in which the amino acid identifier sequences aligned was supported by the key amino acid identifier sequences are selected. For example, a key amino acid identifier sequence presents as RHS, if a situation of xRHSxx is present in an aligned region, the aligned region is considered to be supported by the key amino acid identifier sequence. After acquiring alignment records supported by key amino acid identifier sequences, alignment records of different short peptides need to be integrated to generate epitope regions.

### 6. Generation of Spatial Alignment Regions and Identification of Major Binding Sites

Firstly, the short peptides and the spatial alignment records are subjected to multi-keyword reverse sorting (ranking in descending order) according to the first signal intensity values and the spatial alignment scores of the short peptides, followed by reverse sorting according to the spatial alignment score of each of the spatial alignment records corresponding to each of the strong signal short peptides. The short peptide at the first place was selected according to the ranking order, and the aligned sites in the short peptide are taken as seeds, and these seeds are defined as the sites in the existing region; then the spatial alignment records of other unused strong signal short peptides are iterated over; after the expansion of the existing region, all the alignment records of all short peptides are iterated over again due to the increase of sites comprised in the existing region, including other spatial alignment records corresponding to the first short peptide. If a short peptide presented in the spatial alignment record of which the number of sites comprised in the existing region is more than the number of sites outside the region, then all the sites in the aligned record are added into the existing region sites, results in the expansion of the existing region. Iterating in turn until the sites in the existing region are no longer increased. Among them, there will be some strong signal short peptides with all corresponding spatial alignment records that do not comprising sites in the existing region. These strong signal short peptides will be used to replace unused strong signal short peptides and their seeds according to the above ranking order to form new existing regions, and iterated continuously until no new existing regions were generated. All the existing regions which completed expansion formed a space alignment region together, with each region had short peptides and alignment records used for forming the region, and the sites supported by the key amino acid identifier sequences in any alignment record among the sites comprised in the region were selected as the key binding site. The accumulation of the first signal intensity value of each of the strong signal short peptides at each binding site is the cumulative score of the site.

In one aspect, the present disclosure provides a method for identifying an antigenic epitope, comprising: acquiring a set of amino acid identifier sequences that bind to an antibody to be detected and generate signals in a polypeptide chip, and generating a set of key amino acid identifier sequences based on the set of amino acid identifier sequences;
generating a spatially alignment region based on the position of a short peptide that binds to the antibody to be detected and generates a strong signal in the polypeptide chip in the spatial structure of targeted protein;
identifying key binding sites of the antigenic epitope comprised in the spatially alignment region based on the spatially alignment region.

In some embodiments, acquiring a set of the amino acid identifier sequences that bind to an antibody to be detected and generate signals in a polypeptide chip comprises:
for the short peptide in the polypeptide chip, determining a first signal intensity value of the short peptide according to signal intensity value of signal generated after the short peptide binds to the antibody to be detected, wherein the first signal intensity value is within a preset range;
determining a set of signal short peptides among the short peptides in the polypeptide chip based on the first signal intensity values of the short peptides in the polypeptide chip; and
generating the set of amino acid identifier sequences based on the amino acid sequence of each of the signal short peptides.

In some preferred embodiments, determining a set of signal short peptides among the short peptides in the polypeptide chip based on the first signal intensity values of the short peptides in the polypeptide chip comprises:
determining the short peptides among each of the signal short peptides having first signal intensity values satisfying at least one of the following conditions as the set of signal short peptides:
first condition: the first signal intensity value is the difference between the logarithm of the signal intensity value of the short peptide divided by and the logarithm of the reference signal intensity value, and the first signal intensity value >1;
second condition: the first signal intensity value is a ratio of the signal intensity value of the short peptide divided by a reference signal intensity value, wherein the first signal intensity value is greater than a preset proportion threshold;
third condition: sorting the first signal intensity value of each of the short peptides in descending order, wherein the first signal intensity values are ranked within a preset ranking range.

In some more preferred embodiments, the signal short peptide is a short peptide satisfying both the first condition and the third condition.

In some more preferred embodiments, the signal short peptide is a short peptide satisfying both the second condition and the third condition.

In some more preferred embodiments, the signal short peptide is a short peptide with the first signal intensity value satisfying one or more of the following conditions:
(1) the first signal intensity value is the difference between the logarithm of the signal intensity value of the short peptide divided by and the logarithm of the reference signal intensity value, and the first signal intensity value>1;
(2) the first signal intensity value is a ratio of the signal intensity value of the short peptide and a reference signal intensity value, and the signal value of the short peptide is more than twice of the reference signal value;
(3) sorting the first signal intensity value of each of the short peptides in descending order, wherein the first signal intensity values are ranked at the first 500-2000 places.

In some more preferred embodiments, the reference signal is selected from a blank group signal or a background signal, wherein the blank group signal is a signal of a reserved short peptide which is not combined with the antibody to be detected, and the background signal is a signal which is common in the use of the short peptide.

In some more preferred embodiments, the signal short peptide is the short peptides with the first signal intensity values ranked at the first 2000 places after sorting the first short peptides with signal intensity value >1 in descending order, or the short peptides with first signal intensity values >1 among the first 2000 short peptides after sorting the signal intensity values in descending order.

In some more preferred embodiments, the signal short peptide is the short peptides with the first signal intensity values ranked at the first 2000 places after sorting the first short peptides with signal value more than twice of the reference signal value in descending order, or the short peptides with signal value more than twice of the reference signal value among the first 2000 short peptides after sorting the signal intensity values in descending order.

In some embodiments, generating a set of key amino acid identifier sequences based on the set of amino acid identifier sequences comprises:
generating a set of amino acid identifier subsequences according to the set of amino acid identifier sequences;
screening the set of amino acid identifier subsequences and acquiring a set of binding amino acid identifier sequences; and
screening the set of binding amino acid identifier sequences and acquiring a set of key amino acid identifier sequences.

In some embodiments, generating set of amino acid identifier subsequences according to the set of amino acid identifier sequences comprises:
for each amino acid identifier sequence in the set of amino acid identifier sequences, performing the following segmentation operation and generating a set of first subsequences: segmenting the amino acid identifier sequence into first subsequences comprising preset numbers of amino acid identifiers, wherein the amino acid identifiers in the first subsequence appear continuously in the amino acid identifier sequence;
for each amino acid identifier sequence in the set of amino acid identifier sequences, performing a space addition operation and generating a set of second subsequences, wherein the second subsequences are amino acid identifier subsequences comprising preset numbers of amino acid identifiers and the amino acid identifiers in the second subsequence appear non-continuously in the amino acid identifier sequence;
adding the set of first subsequences and the set of second subsequences into the set of amino acid identifier subsequences to generate the set of amino acid identifier subsequences;
wherein performing a space addition and generating a set of second subsequences comprises:
setting that the first and last positions of each amino acid identifier sequence in the set of amino acid identifier sequences are not spaced, calculating whether the number of remaining amino acid identifiers in the second subsequence generated after space addition is greater than or equal to a preset space addition number, wherein the number of remaining amino acid identifiers is the number of remaining amino acid identifiers excluding the first and last amino acid identifiers;
if yes, performing a space addition operation on the full amino acid identifier subsequence composed of amino acid identifiers, wherein the space addition operation comprises:
replacing amino acid identifiers in the full amino acid identifier subsequence one by one using from 1 to n designated placeholders to generate a second subsequence with n spaces added;
where n is a preset number of spaces added;
if no, performing an amino acid identifier addition operation on an all-empty subsequence formed by the designated placeholder, wherein the amino acid identifier addition operation comprises:
replacing designated placeholders one by one using from 1 to L−2−n amino acid identifiers to generate a second subsequence comprising L−2−n remaining amino acid identifiers;
wherein L is a preset length of the second subsequence, and n is a preset number of spaces added.

In some preferred embodiments, the set of amino acid identifier subsequences comprises amino acid identifier subsequences of less than or equal to 13 amino acids in length.

In some more preferred embodiments, the set of amino acid identifier subsequences comprises amino acid identifier subsequences of less than or equal to 6 amino acids in length.

In some embodiments, screening the set of amino acid identifier subsequences and acquiring a set of binding amino acid identifier sequences comprises:
acquiring second signal intensity value of each of the amino acid identifier subsequences in the set of amino acid identifier subsequences, wherein the second signal intensity value is an average value of signal intensity of the signal intensity values ranked within a preset ranking range among the signal intensity values of short peptides corresponding to the amino acid identifier subsequences sorted in descending order;
acquiring enrichment analysis values of each of the amino acid identifier subsequences, wherein the enriched analysis value is used for characterizing a value of randomness of an amino acid identifier subsequence appearing in a signal short peptide;
determining whether the second signal intensity values and the enrichment analysis values of each of the amino acid identifier subsequences are greater than a preset threshold value;
if yes, the amino acid identifier subsequence is a binding amino acid identifier sequence;
if no, the amino acid identifier subsequence is not a binding amino acid identifier sequence.

In some preferred embodiments, the enrichment analysis value is Zscore, wherein acquiring the Zscore comprises:
randomly sampling from all short peptides in the polypeptide chip, and extracting a preset number of short peptides each time;
repeating the random sampling for a preset number of times;
generating a set of amino acid identifier subsequences of the acquired short peptides after each sampling; counting the number of short peptides comprising the amino acid identifier subsequence in each sampling to obtain the distribution of the number of short peptides comprising the amino acid identifier subsequence over the preset number of times, and acquiring an average value mean and a standard deviation sd; performing polypeptide chip detection on a sample to be detected to obtain the signal short peptide, generating a set of amino acid identifier subsequences based on the signal short peptide, counting the number of short peptides comprising a certain amino acid identifier subsequence, marked as X;
wherein the number of the extracted preset number of short peptides should be equal to the number of the signal short peptides obtained when the sample to be tested is subjected to the polypeptide chip detection; Zscore is calculated and acquired according to the following formula

$$Zscore = \frac{X - mean}{sd}$$

In some more preferred embodiments, the preset sorting range is the three short peptides ranked at the first places.

In some more preferred embodiments, the preset number of times is 10000, and the preset number of short peptides per sampling is 2000.

In some preferred embodiments, the amino acid identifier subsequence is a binding amino acid subsequence when the second signal intensity value for the amino acid substring is >3 and the enrichment assay value is >10.

In some embodiments, screening the set of binding amino acid identifier sequences and acquiring a set of key amino acid identifier sequences comprises:
for each binding amino acid identifier sequence in the set of binding amino acid identifier sequences, the number responsive to the amino acid identifiers in the binding amino acid identifier sequence being equal to m, wherein m is a positive integer, the binding amino acid identifier sequence is a first binding amino acid identifier sequence, and performing the following recursive filtering operation, comprising:
determining whether the second signal intensity value or enrichment analysis value of a second binding amino acid identifier sequence is larger than that of the first binding amino acid identifier sequence, wherein the second binding amino acid identifier sequence is a binding amino acid identifier sequence in the set of the binding amino acid identifier sequences, the amino acid identifier sequence corresponding to the second binding amino acid identifier sequence can generate the first binding amino acid identifier sequence, and the number of amino acid identifiers in the second binding amino acid identifier sequence is m+1;
if yes, the second binding amino acid identifier sequence is a key amino acid identifier sequence;
if no, the second binding amino acid identifier sequence is not the key amino acid identifier sequence.

In some more preferred embodiments, m is 1.

In some embodiments, being based on the position of a short peptide that binds to the antibody to be detected and generates a strong signal in the polypeptide chip in the spatial structure of targeted protein comprises:
for the short peptide in the polypeptide chip, determining a first signal intensity value of the short peptide according to signal intensity value of signal generated after the short peptide binds to the antibody to be detected, wherein the first signal intensity value is within a preset range;
determining a set of strong signal short peptides among the short peptides in the polypeptide chip based on the first signal intensity values of the short peptides in the polypeptide chip;
aligning each of the strong signal short peptides in the set of strong signal short peptides to the spatial structure of a target protein to generate a set of spatial alignment records corresponding to each of the strong signal short peptides, wherein each of the strong signal short peptides corresponds to at least one spatial alignment record;
screening each of the spatial alignment records in the set of spatial alignment records, and determining whether the amino acid identifier sequences of the strong signal short peptides corresponding to each of the spatial alignment records comprise a key amino acid identifier sequence;
if yes, add the strong signal short peptide corresponding to the spatial alignment record into a set of the epitope regions to be integrated;
if no, the strong signal short peptide corresponding to the spatial alignment record is not added into a set of the epitope regions to be integrated.

In some more preferred embodiments, the strong signal short peptide is a short peptide with the first signal intensity value satisfying one or more of the following conditions:
(1) the first signal intensity value is the difference between the logarithm of the signal intensity value of the short peptide divided by and the logarithm of the reference signal intensity value, and the first signal intensity value >3;
(2) the first signal intensity value is the difference between the signal intensity value of the short peptide and the reference signal intensity value, and the signal value of the short peptide is more than 8 times of the reference signal value;
(3) sorting the first signal intensity value of each of the short peptides in descending order, wherein the first signal intensity values are ranked at the first 100 places.

In some more preferred embodiments, the signal short peptide is the short peptides with the first signal intensity values ranked at the first 100 places after sorting the first short peptides with signal intensity value >3 in descending order, or the short peptides with first signal intensity values >3 among the first 100 short peptides after sorting the signal intensity values in descending order.

In some more preferred embodiments, the signal short peptide is the short peptides with the first signal intensity values ranked at the first 100 places after sorting the first short peptides with signal value more than 8 times of the reference signal value in descending order, or the short peptides with signal value more than 8 times of the reference signal value among the first 100 short peptides after sorting the signal intensity values in descending order.

In some preferred embodiments, the alignment adopts an alignment software.

In some preferred embodiments, the alignment software is selected from PepSurf, Pep-3D-Search, and PepMapper.

In some more preferred embodiments, the alignment software is PepSurf.

In some more preferred embodiments, the number of iterations of the alignment does not exceed 100000.

In some preferred embodiments, the spatial alignment record is significant (P value <0.05).

In some embodiments, generating the spatially alignment region comprises:
each of the strong signal short peptides in the set of the epitope regions to be integrated corresponds to a first signal intensity value;
sorting each of the strong signal short peptides by the first signal intensity values corresponding to each of the strong signal short peptides in the set of the epitope regions to be integrated being ranked in descending order;
for each of the sorted strong signal short peptides, performing the following region growth operation according to the ranking order of the strong signal short peptide:
for each of the spatial alignment records in the set of spatial alignment records corresponding to the strong signal short peptide, sorting each of the spatial alignment records of the short peptide by ranking spatial alignment score of each of the spatial alignment records in descending order;
for each of the sorted spatial alignment records corresponded the strong signal short peptides, according to the ranking order of each of the spatial alignment records, taking the amino acid site that aligned with the target antigen in the first-ranking spatial alignment record as a seed, adding the seed into an existing region, wherein the existing region is an intermediate state region before completing the region growth operation;
based on the existing region, selecting a strong signal short peptide and each of its corresponded spatial alignment records from each of the sorted strong signal short peptides and each of the corresponded sorted spatial alignment records according to the ranking order of each of the strong signal short peptides, and performing the following existing region growth operation:
determining whether the site in the spatial alignment record comprises a site in the existing region, and the number of sites comprised in the existing region is not less than the number of sites not comprised in the existing region;
if yes, adding sites not comprised in the existing region in the spatial alignment record into the existing region;
if no, performing the region growth operation on the next spatial alignment record corresponding to the strong signal short peptide or the next strong signal short peptide by the ranking order of each of the spatial alignment records corresponding to the strong signal short peptide or the ranking order of each of the strong signal short peptides;
if the sites in the existing region increases after iterating over the strong signal short peptides for once, iterating over all the strong signal short peptides again until the sites in the existing region no longer increase, and completing the region growth operation;
for each of the sorted spatial alignment records corresponding to the strong signal short peptide, determining whether at least one spatial alignment record in all spatial alignment records corresponding to the strong signal short peptide comprises a site in a first existing region;
if no, adding the strong signal short peptide and the corresponding ranking order thereof into a set of candidate seeds;
for each of the strong signal short peptides in the set of candidate seeds, performing the existing region generation operation and the existing region growth operation according to the ranking order corresponding to each of the strong signal short peptides;
updating the set of candidate seeds after the existing region growth operation completed until no new seed can be screened;
acquiring all existing regions which have completed the growth, adding the sites in each existing region into a set of spatially alignment regions, and generating the spatially alignment region.

In some embodiments, identifying key binding sites of the antigenic epitope comprised in the spatially alignment region based on the spatially alignment region comprises:
determining whether the amino acid site is comprised in a key amino acid identifier sequence in any of corresponding spatial alignment records based on amino acid sites aligned with the target antigen in the spatial alignment region;
if yes, the amino acid site is a key binding site of epitope in the targeted protein.

In another aspect, the present disclosure provides a method for generating an amino acid identifier sequence with a specific length, comprising:
generating an amino acid identifier sequence corresponding to an amino acid sequence, performing the following segmentation operation and generating a set of first subsequences: segmenting the amino acid identifier sequence into first subsequences comprising preset numbers of amino acid identifiers, wherein the amino acid identifiers in the first subsequence appear continuously in the amino acid identifier sequence;
for each amino acid identifier sequence in the set of amino acid identifier sequences, performing a space addition operation and generating a set of second subsequences, wherein the second subsequences are amino acid identifier subsequences comprising preset numbers of amino acid identifiers and the amino acid identifiers in the second subsequence appear non-continuously in the amino acid identifier sequence;
adding the set of first subsequences and the set of second subsequences into the set of amino acid identifier subsequences to generate the set of amino acid identifier subsequences;
wherein performing a space addition and generating a set of second subsequences comprises:
setting that the first and last positions of each amino acid identifier sequence in the set of amino acid identifier sequences are not spaced, determining whether the number of remaining amino acid identifiers in the second subsequence generated after space addition is greater than or equal to a preset space addition number, wherein the number of remaining amino acid identifiers is the number of remaining amino acid identifiers excluding the first and last amino acid identifiers;
if yes, performing a space addition operation on the full amino acid identifier subsequence composed of amino acid identifiers, wherein the space addition operation comprises:
replacing amino acid identifiers in the full amino acid identifier subsequence one by one using from 1 to n designated placeholders to generate a second subsequence with n spaces added;
where n is a preset number of spaces added;
if no, performing an amino acid identifier addition operation on an all-empty subsequence formed by the designated placeholder, wherein the amino acid identifier addition operation comprises:
replacing designated placeholders one by one using from 1 to L−2−n amino acid identifiers to generate a second subsequence comprising L−2−n remaining amino acid identifiers;
wherein L is a preset length of the second subsequence, and n is a preset number of spaces added.

In some preferred embodiments, the set of amino acid identifier subsequences comprises amino acid identifier subsequences of less than or equal to 13 amino acids in length.

In some more preferred embodiments, the set of amino acid identifier subsequences comprises amino acid identifier subsequences of less than or equal to 6 amino acids in length.

The above method proposed in the present disclosure can further identify key binding sites in epitopes that play a key role in antigen antibody binding on the basis of identifying the spatial epitope binding sites.

For the purpose of clarity and brevity of description, features are described herein as part of the same or separate embodiments, however, it will be understood that the scope of the disclosure may include embodiments having combinations of all or some of the features as described.

### EXAMPLES

### Example 1: Method for Identifying Critical Binding Sites for Spatial Epitopes

### 1. Determination of Signal Short Peptides

The signal intensity of monoclonal antibody samples is detected by polypeptide chip. The minimum sample adding unit of the chip is a slide, and each slide is sufficient for repeatedly display of all short peptides in the polypeptide chip. In monoclonal antibody experiments, one set of diluted solutions of the monoclonal antibodies to be tested was added to each slide. In order to evaluate the signal generated by the binding of monoclonal antibodies to short peptides on the chip, space groups were reserved to extract reference signal values. After the signal intensity values of each group was obtained, difference of the logarithmic value of the signal of the monoclonal antibody group and the logarithmic value of the signal of the space group (reference signal value) was calculated, and the first signal intensity value of each short peptide on the chip was obtained as the signal intensity of the short peptide. If no space group is reserved, the generally stable background signal of the short peptides in the chip was adopted as the reference signal value.

The first 2000 short peptides with the first signal intensity values >1 are taken as signal short peptides, indicating that these short peptides on the polypeptide chip can bind to monoclonal antibodies to generate signals; according to the total number of short peptides on the polypeptide chip, the first 500-1000 short peptides with the first signal intensity value >1 can also be taken as signal short peptides. The larger the first signal intensity value, the stronger the signal, indicating that the binding between the short peptide and the antibody is stronger. The short peptides with the first signal intensity value >3 were further defined as strong signal short peptides.

### 2. Splitting of Amino Acid Identifier Subsequences

In order to find out how each peptide binds to the antibody, and by which amino acids and combinations of the amino acids, all amino acid identifier subsequences of length <7 (including the first subsequence consisting of all amino acids without space and the second subsequence spaced by the designated placeholder “.”) were generated from the signal peptide. Each subsequence may comprise 1-6 amino acid (aa) identifiers.

The algorithm for splitting of subsequences is divided into two parts:

From a given sequence, the first subsequences of all lengths were spilted firet, and then all second subsequences were generated by iterating over space addition, wherein the amino acid positions at the first and last of the subsequence are positions that cannot be added with space. For example, number of the amino acid identifier subsequence of length L (i.e., comprising L amino acid identifiers) can be space added is from 1 to L−2. When the number of spaces is n, determine the relative size of the number of remaining amino acids after spacing L−2−n and the number of spaces n. If L−2−n≥n, then the number of remaining amino acid after space addition is more than the number of spaces, and the second subsequence was generated by space adding method; if the number of remaining amino acid after space addition is less than the number of spaces, then the second subsequence was generated by amino acid adding method.

The space adding method refers to a given full amino acid identifier string consisting of amino acids, where the amino acids were replaced by a designated placeholder, such as “.”, to generate second subsequences of various forms with 1 to n spaces added in.

The amino acid adding method refers to a given all-empty string consisting of a designated placeholder, such as “.”, where the “.” were replaced by amino acids given by a specified placeholder, to generate second subsequences of various forms with 1 to L−2−n amino acids added in.

For example, if subsequences of “RHSVV” with two spaces added in were generated, i.e., the length of amino acid identifier subsequence is L=5, the number of spaces is n=2, and the number of spaces is greater than the number of remaining amino acids (L−2−n=1), then the amino acid adding method was used. Finally, subsequences of various forms with one amino acid reserved were generated. For the all-empty string “ . . . ”, there are three possible positions for each one amino acid to be placed in, so three subsequences RH . . . V, R.S.V and R . . . VV were finally generated.

### 3. Definition of Binding Amino Acid Identifier Sequences

(1) Calculate the second signal intensity value of the amino acid identifier subsequences: the binding amino acid identifier sequences correspond to the binding units of the short peptide and the antibody. Firstly, calculate the second signal intensity value of each of the amino acid identifier subsequences generated in the previous step, i.e., the average value of the first signal intensity values of the three short peptides comprising the subsequence with the largest first signal intensity value.

(2) Calculation the Zscore of amino acid identifier subsequences: Zscore of each of the amino acid identifier subsequences was further calculated considering the advantages and limitations of the second signal intensity value. For example, a sample to be tested is subjected to polypeptide chip detection, the first 2000 short peptides with the first signal intensity values >1 are taken as signal short peptides, a set of amino acid identifier subsequences is generated based on the signal short peptides, and the number X of short peptides comprising a certain amino acid identifier subsequence is counted; sample 10000 times randomly from all short peptides covered by the chip, then 2000 short peptides sampled each time, generate all amino acid sub-strings of these short peptides and count the number of short peptides comprising a certain subsequence, and finally obtain the distribution of the number of short peptides comprising the subsequence in the 10000 times, calculate the mean value and standard deviation sd, and calculate Zscore according to the following formula.

$$Zscore = \frac{X - mean}{sd}$$

The Zscore value reflects whether the subsequence appears randomly in the signal short peptides.

A subsequence with larger Zscore results in binding of the short peptide to the antibody, while a larger value of the second signal intensity means that the subsequence results in stronger binding to the antibody. Therefore, in practice, a Zscore >10 with a second signal intensity value >3 is adopted to define the binding amino acid identifier sequence.

(3) Screening of key amino acid identifier sequences: starting from a 1aa subsequence, recursively filter subsequences with amino acid identifier numbers plus 1. For example, for the subsequence RHS, if the Zscore or the second signal intensity value of the RHS is higher than the Zscore or the second signal intensity value of RH, HS and R.S, then the RHS is considered to be a key amino acid identifier sequence; if the Zscore and the second signal intensity are both less than a subsequence with an amino acid length shorter than that of the RHS, such as RH, then the RHS is considered to have an amino acid S added on the basis of RH, which is not beneficial for binding antibodies to epitopes, thus the RHS is not a key amino acid identifier sequence.

### 4. Strong Signal Peptides Alignment to Protein Spatial Structure

Considering the limitation of computational performance, only strong signal short peptides with the first signal intensity value >3 are selected for spatial alignment. The parameter information is “Van del Waals radii sets: Chothia, Probe radius in angstrom:3.9, Calculation mode: Accessible and molecular surface areas”. Then pepSurf was used to align the strong signal short peptides to the spatial structure of the target protein, and the parameter was that the maximum number of iterating cycles should not exceed 100,000. Finally, significant (p<0.05) records of all alignable pairs were obtained. Usually there are 3-5 significant alignment records for each short peptide, so it is necessary to screen the alignment records.

### 5. Screening of Spatial Comparison Results

After the alignment is completed, there are usually multiple alignment results for each short peptide. Alignment records in which the amino acid sequences aligned was supported by the key amino acid identifier sequences are selected. For example, a key amino acid identifier sequence presents as RHS, if a situation of xRHSxx is present in an aligned region, the aligned region is considered to be supported by the key amino acid identifier sequence. After acquiring alignment records supported by key amino acid identifier sequences, alignment records of different short peptides need to be integrated to generate epitope regions.

### 6. Generation of Spatial Alignment Regions and Identification of Major Binding Sites

Firstly, the short peptides and the spatial alignment records are subjected to multi-keyword reverse sorting (ranking in descending order) according to the first signal intensity values and the spatial alignment scores of the short peptides, followed by reverse sorting according to the spatial alignment score of each of the spatial alignment records corresponding to each of the strong signal short peptides. The short peptide at the first place was selected according to the ranking order, and the aligned sites in the short peptide are taken as seeds, and these seeds are defined as the sites in the existing region; then the alignment records of other unused strong signal short peptides are iterated over; after the expansion of the existing region, all the alignment records of all short peptides are iterated over again due to the increase of sites comprised in the existing region, including other spatial alignment records corresponding to the first short peptide. If a short peptide presented in the spatial alignment record of which the number of sites comprised in the existing region is more than the number of sites outside the region, then all the sites in the aligned record are added into the existing region sites, results in the expansion of the existing region, and the short peptide is marked as used. Iterating in turn until the sites in the existing region are all no longer increased when adding any strong signal peptide. Among them, there will be some strong signal short peptides with all corresponding spatial alignment records that do not comprising sites in the existing region. These strong signal short peptides will be used to replace unused strong signal short peptides and their seeds according to the above ranking order to form new existing regions, and iterated continuously until no new existing regions were generated. All the existing regions which completed expansion formed a space alignment region together, with each region had short peptides and alignment records used for forming the region, and the sites supported by the key amino acid identifier sequences in any alignment record among the sites comprised in the region were selected as the key binding site. The accumulation of the first signal intensity value of each of the strong signal short peptides at each binding site is the cumulative score of the site.

### Example 2: Binding of Antibodies to be Tested to Polypeptide Chips

### 1. Reagents and Chips Used in the Example

In this example, 17 kinds of purchased monoclonal antibodies with known epitopes (see Table 1 for details of product information) were used, and the V16 polypeptide chips (Item No.: V16_10296) of Health Tell Company were used to carry out the antibody and chip binding experiment according to standard experimental procedures.

TABLE 1
Manufacturer Nos. and known epitopes of the purchased antibodies

| | | | | Binding Sites in Spatially |
|---|---|---|---|---|
| Manufacturer | Product No. | Clone No. | Known Epitope | Resolved Structures |
| absolute- | Ab00805-1.1 | F11.2.32 | O90777_9PLVG:36-46:MSLPGRWKPKM | 36; 37; 38; 39; 40; 41; 42; 43; 44; 45 |
| antibody | | | | |
| absolute- | Ab00120-1.1 | 6A7 | BAX_HUMAN:13-19:PTSSEQI | 13; 14; 15; 16; 17; 18; 19 |
| antibody | | | | |
| absolute- | Ab00100-1.1 | 9E10 | MYC_HUMAN:410-419:EQKLISEEDL | 410; 411; 412; 413; 414; 415; 416; 417; |
| antibody | | | | 418; 419; 420 |
| absolute- | Ab00339-1.4 | a.4.6.1 | VEGFA_HUMAN:F43; Y47; Y71; | 25; 45; 48; 63; 65; 66; 79; 80; 81; 82; |
| antibody | | | K74; Q105; I106; M107; R108; | 83; 84; 85; 86; 87; 88; 89; 90; 91; 92; |
| | | | I109; K110; H112; Q113; G114; | 93; 94; 103; 104; 105; 106 |
| | | | Q115; H116; I117; G118; E119; M120 | |
| absolute- | Ab00917- | e111 | Q8BE40_9FLAV:S338; T339; | 299; 300; 301; 302; 303; 304; 309; 310; |
| antibody | 10.0 | | Q340; D341; E342; K343; G344; | 313; 314; 315; 316; 317; 318; 319; |
| | | | A345; T346 | 321; 323; 325; 328; 329; 330; 332; |
| | | | | 334; 335; 336; 337; 338; 339; 340; |
| | | | | 342; 343; 344; 345; 346; 347; 348; 349; |
| | | | | 350; 351; 352; 353; 354; 355; 357; |
| | | | | 359; 361; 362; 363; 364; 366; 368; |
| | | | | 369; 370; 371; 372; 373; 374; 375; |
| | | | | 377; 379; 381; 382; 383; 384; 385; 386; |
| | | | | 388; 390; 391; 392; 393; 394; 395 |
| absolute- | Ab00124- | Campath-1H | CD52_HUMAN:1-14:GQNDTSQTSSPSAD | 7, 8, 9, 10, 11, 12, 13, 14 |
| antibody | 10.0 | | | |
| absolute- | Ab00718- | D2E7 | TNFA_HUMAN:P20, Q21, E23, | 6; 7; 8; 9; 10; 20; 21; 23; 24; 27; 30; 31; |
| antibody | 10.0 | (Adalimumab) | K65, Q67, E110; P113, Y141, | 37; 38; 39; 40; 42; 43; 44; 45; 46; 49; |
| | | | A145; E146, T72, H73, T77, T79, S81, | 65; 66; 67; 72; 73; 74; 75; 77; 79; 81; |
| | | | K90, N92; E135, N137 | 85; 88; 90; 91; 92; 97; 110; 111; 112; |
| | | | | 113; 115; 128; 131; 135; 136; 137; 138; |
| | | | | 140; 141; 142; 143; 144; 145; 146; |
| | | | | 147 |
| absolute- | Ab00715- | Bevacizumab | VEGFA_HUMAN:45; 48; 79; 80; | 25; 45; 48; 63; 65; 66; 79; 80; 81; 82; |
| antibody | 10.0 | | 81; 82; 83; 84; 86; 87; 88; 89; 90; 91; | 83; 84; 85; 86; 87; 88; 89; 90; 91; 92; |
| | | | 92; 93; 93 | 93; 94; 103; 104; 105; 106 |
| Biovision | A2144 | Canakinumab | IL1B_HUMAN:E64, K65, N66; | 14; 15; 16; 19; 20; 21; 22; 23; 24; 25; |
| | | | D35, Q38, Q34, E37, N129, K27, | 27; 29; 30; 31; 32; 33; 34; 35; 36; 37; |
| | | | N129, S21, E25, E64, E37, K65 | 38; 39; 40; 41; 50; 51; 52; 53; 54; 55; |
| | | | | 63; 64; 65; 66; 67; 86; 87; 88; 97; 117; |
| | | | | 118; 119; 120; 126; 127; 128; 129; 140; |
| | | | | 141 |
| Biovision | A2142 | Certolizumab | TNFA_HUMAN:G24, D45, Q47, | 23; 24; 25; 44; 45; 47; 49; 65; 70; 71; |
| | | | T77, I83, V85, S86, Q88, T89, K90, | 72; 73; 75; 77; 79; 81; 83; 85; 86; 87; |
| | | | R131, E135, N137, R138, P139, | 88; 89; 90; 97; 115; 128; 131; 135; 136; |
| | | | D140 | 137; 138; 139; 140; 145; 146; 149 |
| | | | | 24; 44; 45; 47; 72; 86; 87; 88; 131; 135; |
| | | | | 137; 138; 146 |
| Biovision | A2141 | Golimumab | TNFA_HUMAN:G24, K65, Q67, | 21; 22; 23; 24; 25; 65; 66; 67; 68; 69; |
| | | | S71, E104, T105, P106, E107, G108, | 70; 71; 72; 74; 88; 89; 104; 105; 106; |
| | | | A111, R138, D140, Y141 | 107; 108; 109; 110; 111; 112; 113; 138; |
| | | | | 139; 140; 141; 142; 143; 144 |
| Biovision | A1307 | Nivolumab | PDCD1_HUMAN:L25, D26, S27, | 25; 26; 27; 28; 29; 30; 31; 59; 60; 61; |
| | | | P28, D29, R30, P31; K131, A132, | 62; 72; 73; 75; 115; 127; 128; 129; 130; |
| | | | A129, P130, L128 | 131; 132 |

### 2. Antibody-Chip Binding Experiment

The polypeptide chips used in the examples of the present disclosure is the V16 version chip of HealthTell Company, which contains 3,218,577 short peptides. The chip covers all 4aa combinations of 18 amino acids (excluding cysteine (Cys, C) and methionine (Met, M)), and 99.97% of the 4aa marker subsequences reached coverage of 10 short peptides or more.
(1) Sample preparation: antibody sample was diluted twice with 100 D-mannitol solution to obtain 100 ng/mL sample plate for later use;
(2) Preparation of blocking solution: 7.2 mL of sample diluent was taken into 15 mL centrifuge tube, before addition of 0.8 ml 1% Casein with shaken and well-mixed;

### (3) Plate Layout and Sample Addition

a. Block of the chip: transfer 300 μL of the prepared blocking solution to each well well position of the chip (the chip is placed in Cassette), seal the film, mix at 600 rpm for 20 s, and then incubate at 37 °C in a thermostat for 1 hour;
b. Sample addition of blocked Cassette (chip): The minimum sample addition unit of the chip is a slide, one sample in each slide. In monoclonal antibody experiment, add a group of diluted dissolution solution of monoclonal antibody to each slide, and transfer 300 μL diluted antibody to the corresponding well well position of the Cassette according to the well position information in the plate arrangement table;
(4) First incubation: the Cassette was sealed with film, and placed onto the incubation module of automatic instrument, and incubated for 1 h;
(5) First plate wash: tear the film from the Cassette and wash the plate in automatic plate washer (BioTek®405LS microplate magnetic plate washer);
(6) Adding secondary antibody: under condition of avoiding light (turning off the light), add 2 μL of goat-mouse secondary antibody into 6 mL of secondary antibody diluent (3000-fold dilution), shake and mix well, add 6 μL of goat-human secondary antibody into 9 mL of secondary antibody diluent (1500-fold dilution), and add 550 μL to corresponding wells of Cassette respectively;
(7) Second incubation: the Cassette was sealed with film, and placed onto a constant temperature mixer and incubated for 60 min;
(8) Second plate wash: tear the film from the Cassette amd place it in automatic plate washer, and wash the plate in automatic plate washer (BioTek®405LS microplate magnetic plate washer);
(9) Imaging scanning: Disassemble, clean and dry the chips in the Cassette, assemble them into Imaging Cassette, and put them into ImageXpress micro4 imager of Molecular Device Company for scanning and imaging. A TIFF image file was eventually obtained for each detection sample as the original data.
(10) Data processing:

Logarithmic transformation and standardization were performed on the sample values, and a standardized matrix was output, specifically comprising:
a. Fluorescence intensity values of features extracted, outputting 1 GPR5 data file and 1 image location file, wherein the GPR5 file contains all information about a sample and fluorescence intensity information for all features.
b. Fluorescence intensity information of features extracted from the GPR5 data file for all samples generated a raw fluorescence intensity (FG, foreground) data matrix. Then, logarithmic transformation was performed on the data of each sample to obtain LFG (log-transferred round) data matrix, and Zscore standardization was performed to obtain NLFG (normalized and log-transferred round) data matrix. This step also generated a sample chip information file that includes information such as the sample array location, No. of the chip used, etc.

The data generated in this step (i.e. signal values of antibodies binding to polypeptide chips) was subjected to difference calculation between logarithm values of the signal of monoclonal antibody group and the signal of space group to obtain first signal intensity value of each short peptide on chip, and then signal short peptide was obtained by screening with first signal intensity values >1, in order to enter subsequent epitope identification step.

### Example 3: Accuracy Assessment of the Identification of Major Binding Sites of Spatial Epitopes

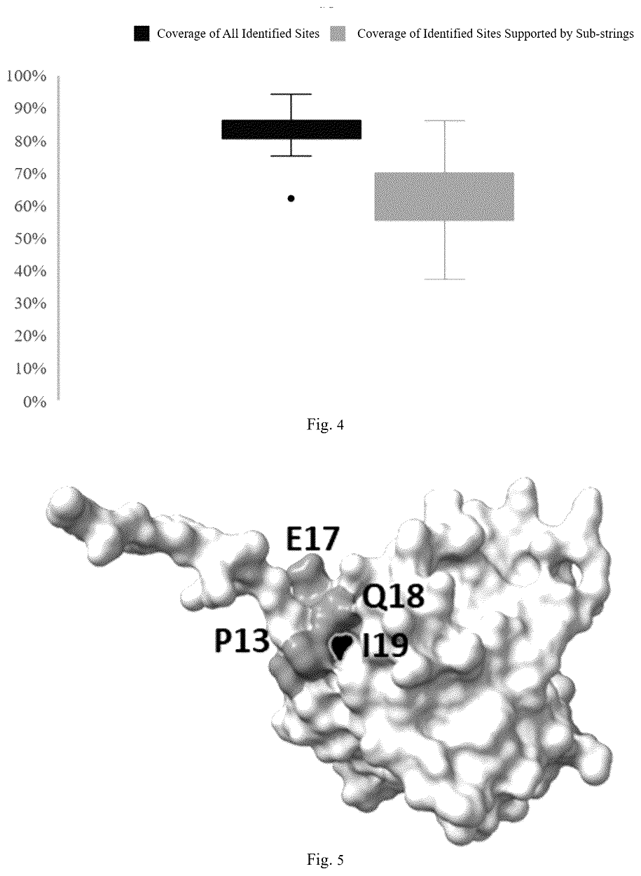

All antibodies with well predicted binding were identified for their spatial epitopes. The identification results are shown in Table 2 and FIG. 4, wherein the “number of known binding sites” is the number of binding sites estimated from the spatial resolution structure of the antibody-antigen or antibody-short peptide complex; the “coverage of all identified sites” is the percentage of known binding sites covered by sites identified using the spatial epitope method of the present disclosure; the “coverage of identified sites supported by subsequence” is the percentage of of known binding sites covered by sites supported by key amino acid identifier sequences, i.e., sites considered to be critical binding sites identified using the spatial epitope method of the present disclosure.

The spatial epitope of an antibody consists of a series of amino acid sites that participate in antigen-antibody binding. In general, that coverage rate of all binding sites identified by the method to binding sites in each antibody spatial epitope can reach 80%, and wherein the coverage rate of sites supported by key amino acid identifier sequences (i. e., key binding sites) to known binding sites can reach 60%. Among them, the linear epitope site of antibody 6A7 and the spatial epitope site of antibody Golimumab were identified well, while the e111 antibody targeting dengue virus was identified poorly for binding sites.

The fluctuation of identification effect of different antibodies was mainly affected by the following factors: 1) The binding intensity of different binding sites in antibody spatial epitopes was different. Different sites form van der Waals forces, hydrogen bonds and other different forces with specific amino acids in antibodies, thus forming binding. The exposure areas of each site on the surface of antigen space structure are different, the area covered in the binding region after binding of antigen and antibody is also different, and the type of binding forces they depend on are also different, so the strength of the force formed is different; 2) the aggregation degree of strong binding sites. Since the binding strength of antibody spatial epitope sites is different, the spatial epitope identification method of the present disclosure mainly considers sub-strings formed by various combinations of 4 amino acids within the length of 6 amino acids. Therefore, if the distance between strongly binding amino acids on the antigen surface exceeds 6 amino acids, the binding substring signal may not be observed. 3) The limitation of spatial structure. The spatial structures used in the present disclosure are all spatial structures predicted by alphafold2, which may deviate from the real structure. 4) Conformational changes caused by antigen-antibody binding. In the epitope, there are some sites that may play a binding role before the structural change, but after the spatial conformation change caused by binding of antigen and antibody, the effect is relatively weakened. This change results in the binding sites identified are binding sites before conformational changes (assuming that binding of the peptide to the antibody does not cause conformational changes in the antibody) rather than the structurally resolved binding site after conformational changes.

Three main cases are emphatically analyzed below:

The known spatial epitope of 6A7_Bax antibody consists of 7 binding sites, which is obtained from the spatial structure analysis of the short peptide PTSSEQI and 6A7 antibody binding complex composed of 7 sites of the short peptide. When a short peptide with strong antibody binding signal is aligned to the spatial structure predicted by alphafold2 of Bax, the seventh Ile amino acid in short peptide PTSSEQI has a very small area on the spatial surface of the protein (as shown in FIG. 5), so it is not considered to be able to be aligned to this position during spatial alignment, so this amino acid cannot be identified by spatial epitope method. Only 6 binding sites were identified (wherein Q18 had the lowest relative score) by the present spatial epitope method. This also suggests that although a specific amino acid can bind to the antibody in the synthesized short peptide and enhance the binding capacity of the short peptide, it may not directly participate in the binding when the complete antigen protein binds to the antibody in the spatial structure, i.e., it may not be a key binding site. It is also possible that conformational changes occur when the antibody binds to the intact Bax antigen protein, which further exposes the Ile amino acids to the spatial surface and allows them to participate in binding with the antibody. In the present method, 6 amino acid sites were identified as key binding sites of spatial epitopes.

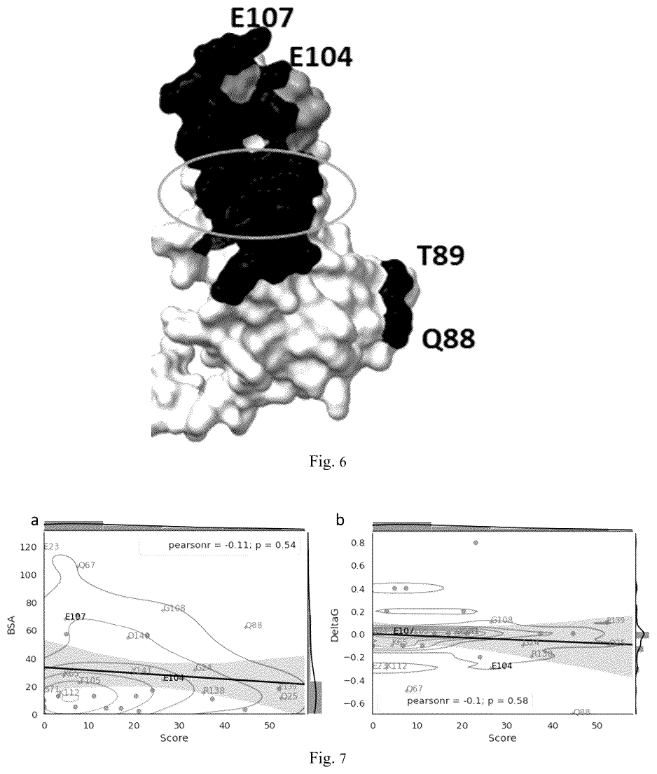

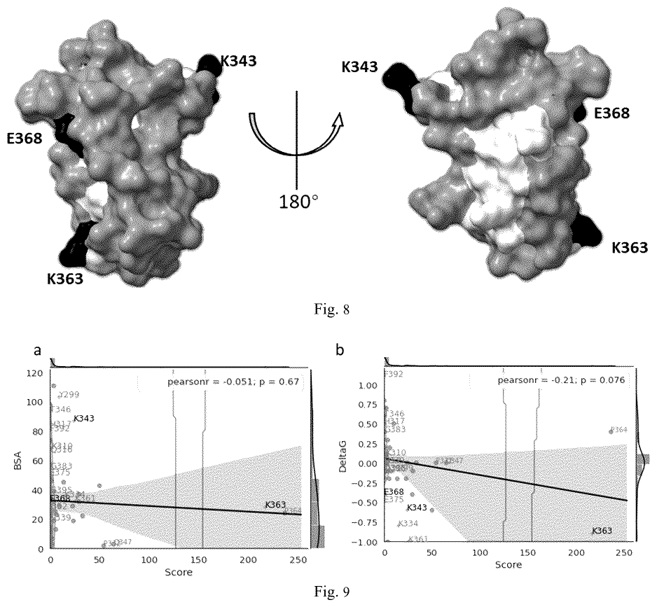

The antibody spatial epitope of Golimumab consists of 33 amino acids on TNF. These 33 amino acids form two binding regions (shown in FIG. 6), wherein 88 and 89 forming one region and the other binding sites forming the other. The spatial epitope identification method identified both regions and most sites. Among these sites, E104 and E107 are considered in the resolved spatial structure to be capable of forming hydrogen bonds and salt bridges and are important sites that distinguish this antibody from other TNF monoclonal antibodies (Ono, M., et al., Structural basis for tumor necrosis factor blockade with the therapeutic antibody golimumab. Protein Science, 2018. 27(6): p. 1038-1046). Both sites were accurately identified as key binding sites in this method. In addition, the analysis found that the middle binding region formed by G24, Q25, F144 and P139 had the highest binding score, which should play an important role in the antigen-antibody binding process. These sites have not received special attention in structural elucidation studies, which may be due to differences between short peptide-antibody binding and intact antigen-antibody binding, one of the important differences of which being that antigen-antibody binding leads to conformational changes, whereas the technology of the present disclosure relies on short peptide-antibody binding and the binding sites found are before conformational changes.

The spatial epitope of e11 antibody consists of 73 scattered sites on the E protein of dengue virus. Alphafold2 has not published the spatial structure of the protein yet, and no independent spatial structure of the protein has been resolved. Only the spatial structure of the antibody binding complex in the 299-395 region of E protein has been published. Thus, the spatial structure of protein E as used in this disclosure is the structure of a partial region of protein E isolated from the complex. This is one of the main reasons for the low resolution coverage of e111 binding site. Another reason is that the currently known epitopes cover almost the entire spatial structure, which is a great challenge for current epitope identification algorithms. The S338, G344 and V345, which bind to antibody light and heavy chains and form an epitope binding region with K343 reported by literature (Austin, S. K., et al., Structural Basis of Differential Neutralization of DENV-1 Genotypes by an Antibody that Recognizes a Cryptic Epitope. PLoS Pathogens, 2012. 8(10): p. e1002930.) were all identified. Among them, K343 is believed to be able to form hydrogen bonds and salt bridges with the corresponding amino acids of the antibodies, and has the relatively largest BSA. K363 has a relatively smallest AG, is also able to form hydrogen bonds and salt bridges, and forms a key amino acid identifier sequence PK with P364, all of which have been accurately identified. Therefore, the spatial epitope identification of e111 is considered to be relatively reliable.

TABLE 2
Coverage of known binding sites by sites identified by the present method

| | Number of | Coverage | Coverage of | |
|---|---|---|---|---|
| | Known | of All | Identified Sites | |
| | Binding | Identified | Supported by | Identified Sites Supported by Subsequences (Ranking in |
| Antibody_Antigen | Sites | Sites | Subsequences | Reverse Order according to Score of the Sites) |
| 6A7_Bax | 7 | 86% | 86% | Region1: |
| | | | | E17; S16; S15; T14; L25; P13; L141; L149; K21; Q18; G156; Q153; Q155 |
| | | | | Region2: W170; G166; E69; Q171; T167; Y164; T169; P168 |
| 9E10_Myc | 11 | 82% | 55% | Region1: |
| | | | | L404; V406; L396; P391; E410; E409; P382; V393; I414; V394; |
| | | | | Q411; I403; S405; E383; L413; E385; Q407; N387; A401; K412; |
| | | | | L377; Q380; E416; A390; A408 |
| Bevacizumab_VEGF | 26 | 88% | 69% | Region1: G |
| | | | | G88; P85; Q89; K84; P28; Q87; P40; R56; N100; R82; G65; K101; |
| | | | | Q98; Q37; P70; P106; F36; G58; R105; G59; N75; Q79; G92; K107; |
| | | | | K48; Q22; D34; Y39; N62; I83; P53; Y45; T31; K108; E42; L97; |
| | | | | R23; S74; D63; I91; H86; H99; E93 |
| | | | | Region2: |
| | | | | G159; P155; P157; Q171; G154; P160; P173; P153; Q174; K177; |
| | | | | R165; H156; N195; P149; K181; E196; R197; R200; S179; S162; |
| | | | | F169 |
| | | | | Region3: P204; R200; N195; K203; N182; R205; R206; D202 |
| Campath-1H_CD52 | 8 | 75% | 50% | Region1: S34; A37; S36; P35 |
| Canakinumab_IL1 | 49 | 86% | 69% | Region1: |
| beta | | | | P23; G22; P131; P91; K27; Q126; S21; N89; Y24; A127; N66; G135; |
| | | | | P87; F133; S84; S125; R98; V85; N129; N119; E25; R4; Q81; |
| | | | | G140; Q39; K109; G49; Q116; F117; K94; K97; K92; G136; A1; |
| | | | | V47; V132; Q48; Q38; P2; Q141; Y68; I143; P118; P78; Q14; R11; |
| | | | | V72; A115; K88; Y90; E83; V19; D86; K77; K65; K74; Y121; V3; |
| | | | | S5; F46; T9; S13; S52; G139; D145; V40; G33; Q32; Q15; W120; |
| | | | | L67; T79; Q149; K93; K138; L80; F150; L82; D142; L31; T137; |
| | | | | T147; E51; K103; K55 |
| Certolizumab_TNF | 36 | 83% | 58% | Region1: |
| | | | | Q25; Q27; P139; R6; N39; G40; P12; Q47; R138; Q21; K128; P51; |
| | | | | P20; K11; R44; P8; R32; R131; A18; Q88; A22; N46; A96; G24; |
| | | | | R2; Q125; F124; F144; T7; Q149; N19; K90; G148; N92; Y87; G129; |
| | | | | K98; R82; Q61; A38; A84; G54; N137; T89; V91; A156; N34; |
| | | | | A35; L120; D10; R31; K65; A145; L29; L36; T79; S52; D140; V17; |
| | | | | V85; T77; S86; S3 |
| | | | | — |
| D2E7-Adalimumab_TNF | 57 | 84% | 56% | Region1: |
| | | | | G40; N39; P12; K11; K128; P51; R6; P8; Q125; Q47; G54; Q88; |
| | | | | R131; R2; G129; K90; R82; Y87; A38; N92; A84; L37; R44; S52; |
| | | | | L94; V13; Q27; G122; G121; T89; S3; N30; N34; R32; L157; S9; |
| | | | | V85; S86; A18; A156; F124 |
| | | | | Region2: |
| | | | | Q25; Q27; N19; P20; P139; G24; Q21; F144; R138; G66; N137; |
| | | | | N46; A18; A22; Q47; R44; Q67; P113; K112; K98; G68; R131; A111; |
| | | | | R32; R103; N30; R31; K65; P100; Q149; T105; G148; V17; L29; |
| | | | | A35; P117; A33; Y151; Q61; Y115; G121; A96; Y119; G122; |
| | | | | T77; D140 |
| F11.2.32_PR-HIV | 10 | 80% | 70% | Region1: G40; R41; L38; W42; D37; P39; P44; K43 |
| | | | | G40; R41; L38; W42; D37; P39; P44; K43 |
| Golimumab_TNF | 33 | 94% | 82% | Region1: |
| | | | | P12; P51; N39; G40; F124; R82; Q47; Q25; P139; G54; Q88; P117; |
| | | | | N137; Q125; F144; P100; R138; P8; G24; R44; R131; E116; G122; |
| | | | | N92; Q27; K128; A38; N19; L142; R32; E42; K90; Q21; N46; P113; |
| | | | | K11; L63; Y141; N34; D140; D10; K98; S52; L37; G121; L43; |
| | | | | I155; P20; G66; G148; G129; T7; N30; V49; Q149; R31; A84; Q67; |
| | | | | E53; L55; D45; Y87; A22; S9; A96; Y59; H15; A111; S95; V91; |
| | | | | R6; K65; W114; A33; I83; T77; R2; A156; A18; L29; E135; R103; |
| | | | | Q102; S86; L157; K112; Y115; T89 |
| | | | | Region2: |
| | | | | P106; E104; G108; P70; K112; Q67; G68; R103; A111; A109; W114; |
| | | | | T105; E110; P113; G66; E107; P100; H73 |
| | | | | Region3: R2; P8; R6; S3; V1; S4; S5; N39 |
| Nivolumab_PDCD1 | 21 | 86% | 62% | Region1: |
| | | | | P28; D29; R30; D26; P31; F24; S27; L25; G22; P21; L128; Q133; |
| | | | | W32; N33; A132; P34; I126; S127; P130; I134; E136 |
| | | | | Region2: |
| | | | | D85; P83; R86; P89; S87; G90; Y68; E84; F82; D77; D92; R94; R69; |
| | | | | N66; Q88; K78; L79; D48; R112; V64; N49; Q91; F95; S109; P72; |
| | | | | Q75; T76; I134 |
| | | | | Region3: R104; N102; D105; P101; S57; N58; S55 |
| | | | | Region4: N49; R112; D48; E46; G47 |
| a.4.6.1_VEGF | 26 | 85% | 65% | Region1: |
| | | | | G58; G65; N62; Y25; P70; G59; P106; Q22; R56; N100; R23; P28; |
| | | | | Q98; Y21; R105; S24; K101; K107; N75; F17; T71; E64; F36; E72; |
| | | | | E73; K16; P40; Q37; Y39; V20; I76; D19; S74; V69; E67; K108; |
| | | | | E103; H99; S50; P49; E30; R110; A111; R112; E42; P53 |
| | | | | Region2: |
| | | | | G159; G154; Q171; P157; N195; P173; P155; P160; P153; R165; |
| | | | | N182; H156; Q174; E193; R186; R190; P204; V170; Q191; A189; |
| | | | | T175; T183; R205; F169; K181; K177; R200; R164; K203; P149; |
| | | | | W150; S162; K166; T198; D202; S179; K188; E196; S151 |
| | | | | Region3: |
| | | | | G120; G122; G124; R119; V118; K121; S117; Q113; Q125; K126; |
| | | | | E114; K116; K123 |
| | | | | Region4: |
| | | | | G88; K84; Q89; R82; P85; Y45; G92; Q87; E44; F36; P40; I83; E42; |
| | | | | I91; H90; N100; E93; Y39; Q37; R56; I43; E38; I76; N75; T77; E72; |
| | | | | P49 |
| e111_E-DIII-DENV | 73 | 62% | 37% | Region1: |
| | | | | P364; K363; P332; V365; Q323; K361; N366; A331; K334; I357; |
| | | | | Y299; K325; E309; R350; N355; A354; L351; T359; L321; K310 |
| | | | | Region2: |
| | | | | Q347; Q340; R350; G349; G344; K343; K388; L389; L387; N348; |
| | | | | Y377; G374 |

1. A method for identifying an antigenic epitope, comprising:
acquiring a set of amino acid identifier sequences that bind to an antibody to be detected and generate signals in a polypeptide chip, and generating a set of key amino acid identifier sequences based on the set of amino acid identifier sequences;
generating a spatially alignment region based on the position of a short peptide that binds to the antibody to be detected and generates a strong signal in the polypeptide chip in the spatial structure of targeted protein;
identifying key binding sites of the antigenic epitope comprised in the spatially alignment region based on the spatially alignment region.

2. The method according to claim 1, wherein said acquiring a set of the amino acid identifier sequences that bind to an antibody to be detected and generate signals in a polypeptide chip comprises:
for the short peptide in the polypeptide chip, determining a first signal intensity value of the short peptide according to signal intensity value of signal generated after the short peptide binds to the antibody to be detected, wherein the first signal intensity value is within a preset range;
determining a set of signal short peptides among the short peptides in the polypeptide chip based on the first signal intensity values of the short peptides in the polypeptide chip; and
generating the set of amino acid identifier sequences based on the amino acid sequence of each of the signal short peptides;
preferably, said determining a set of signal short peptides among the short peptides in the polypeptide chip based on the first signal intensity values of the short peptides in the polypeptide chip comprises:
determining the short peptides among each of the signal short peptides having first signal intensity values satisfying at least one of the following conditions as the set of signal short peptides:
first condition: the first signal intensity value is a ratio of the signal intensity value of the short peptide divided by a reference signal intensity value, wherein the first signal intensity value is greater than a preset proportion threshold;
second condition: sorting the first signal intensity value of each of the short peptides in descending order, wherein the first signal intensity values are ranked within a preset ranking range;
more preferably, the signal short peptide is a short peptide satisfying both the first condition and the second condition.

3. The method according to claim 1, wherein said generating a set of key amino acid identifier sequences based on the set of amino acid identifier sequences comprises:
generating a set of amino acid identifier subsequences according to the set of amino acid identifier sequences;
screening the set of amino acid identifier subsequences and acquiring a set of binding amino acid identifier sequences; and
screening the set of binding amino acid identifier sequences and acquiring a set of key amino acid identifier sequences.

4. The method according to claim 3, wherein said generating a set of amino acid identifier subsequences according to the set of amino acid identifier sequences comprises:
for each amino acid identifier sequence in the set of amino acid identifier sequences, performing the following segmentation operation and generating a set of first subsequences: segmenting the amino acid identifier sequence into first subsequences comprising preset numbers of amino acid identifiers, wherein the amino acid identifiers in the first subsequence appear continuously in the amino acid identifier sequence;
for each amino acid identifier sequence in the set of amino acid identifier sequences, performing a space addition operation and generating a set of second subsequences, wherein the second subsequences are amino acid identifier subsequences comprising preset numbers of amino acid identifiers and the amino acid identifiers in the second subsequence appear non-continuously in the amino acid identifier sequence;
adding the set of first subsequences and the set of second subsequences into the set of amino acid identifier subsequences to generate the set of amino acid identifier subsequences;
wherein said performing a space addition and generating a set of second subsequences comprises:
setting that the first and last positions of each amino acid identifier sequence in the set of amino acid identifier sequences are not spaced, determining whether the number of remaining amino acid identifiers in the second subsequence generated after space addition is greater than or equal to a preset space addition number, wherein the number of remaining amino acid identifiers is the number of remaining amino acid identifiers excluding the first and last amino acid identifiers;
if yes, performing a space addition operation on the full amino acid identifier subsequence composed of amino acid identifiers, wherein the space addition operation comprises:
replacing amino acid identifiers in the full amino acid identifier subsequence one by one using from 1 to n designated placeholders to generate a second subsequence with n spaces added;
where n is a preset number of spaces added;
if no, performing an amino acid identifier addition operation on an all-empty subsequence formed by the designated placeholder, wherein the amino acid identifier addition operation comprises:
replacing designated placeholders one by one using from 1 to L−2−n amino acid identifiers to generate a second subsequence comprising L−2−n remaining amino acid identifiers;
wherein L is a preset length of the second subsequence, and n is a preset number of spaces added.

5. The method according to claim 3, wherein said screening the set of amino acid identifier subsequences and acquiring a set of binding amino acid identifier sequences comprises:
acquiring second signal intensity value of each of the amino acid identifier subsequences in the set of amino acid identifier subsequences, wherein the second signal intensity value is an average value of signal intensity of the signal intensity values ranked within a preset ranking range among the signal intensity values of short peptides corresponding to the amino acid identifier subsequences sorted in descending order;
acquiring enrichment analysis values of each of the amino acid identifier subsequences, wherein the enriched analysis value is used for characterizing a value of randomness of the amino acid identifier subsequence appearing in a signal short peptide;
determining whether the second signal intensity values and the enrichment analysis values of each of the amino acid identifier subsequences are greater than a preset threshold value;
if yes, the amino acid identifier subsequence is a binding amino acid identifier sequence;
preferably, the enrichment analysis value is Zscore, wherein acquiring the Zscore comprises:
randomly sampling from all short peptides in the polypeptide chip, and extracting a preset number of short peptides each time;
repeating the random sampling for a preset number of times;
generating a set of amino acid identifier subsequences of the acquired short peptides after each sampling;
counting the number of short peptides comprising the amino acid identifier subsequence in each sampling to obtain the distribution of the number of short peptides comprising the amino acid identifier subsequence over the preset number of times, and acquiring an average value mean and a standard deviation sd;
performing polypeptide chip detection on a sample to be detected to obtain the signal short peptide, generating a set of amino acid identifier subsequences based on the signal short peptide, counting the number of short peptides comprising a certain amino acid identifier subsequence, marked as X;
wherein the number of the extracted preset number of short peptides should be equal to the number of the signal short peptides obtained when the sample to be tested is subjected to the polypeptide chip detection;
the Zscore is calculated and acquired according to the following formula
$$Zscore = \frac{X - mean}{sd} .$$

6. The method according to claim 3, wherein said screening the set of binding amino acid identifier sequences and acquiring a set of key amino acid identifier sequences comprises:
for each binding amino acid identifier sequence in the set of binding amino acid identifier sequences, the number responsive to the amino acid identifiers in the binding amino acid identifier sequence being equal to m, wherein m is a positive integer, the binding amino acid identifier sequence is a first binding amino acid identifier sequence, and performing the following recursive filtering operation, comprising:
determining whether the second signal intensity value or enrichment analysis value of a second binding amino acid identifier sequence is larger than that of the first binding amino acid identifier sequence, wherein the second binding amino acid identifier sequence is a binding amino acid identifier sequence in the set of the binding amino acid identifier sequences, the amino acid identifier sequence corresponding to the second binding amino acid identifier sequence can generate the first binding amino acid identifier sequence, and the number of amino acid identifiers in the second binding amino acid identifier sequence is m+1;
if yes, the second binding amino acid identifier sequence is a key amino acid identifier sequence.

7. The method according to claim 1, wherein said based on the position of a short peptide that binds to the antibody to be detected and generates a strong signal in the polypeptide chip in the spatial structure of targeted protein comprises:
for the short peptide in the polypeptide chip, determining a first signal intensity value of the short peptide according to signal intensity value of signal generated after the short peptide binds to the antibody to be detected, wherein the first signal intensity value is within a preset range;
determining a set of strong signal short peptides among the short peptides in the polypeptide chip based on the first signal intensity values of the short peptides in the polypeptide chip;
aligning each of the strong signal short peptides in the set of strong signal short peptides to the spatial structure of a target protein to generate a set of spatial alignment records corresponding to each of the strong signal short peptides, wherein each of the strong signal short peptides corresponds to at least one spatial alignment record;
screening each of the spatial alignment records in the set of spatial alignment records, and determining whether the amino acid identifier sequences of the strong signal short peptides corresponding to each of the spatial alignment records comprise a key amino acid identifier sequence;
if yes, add the strong signal short peptide corresponding to the spatial alignment record into a set of the epitope regions to be integrated;
preferably, the alignment adopts an alignment software; preferably, the alignment software is selected from PepSurf, Pep-3D-Search, and PepMapper; more preferably, the alignment software is PepSurf.

8. The method according to claim 1, wherein said generating the spatially alignment region comprises:
each of the strong signal short peptides in the set of the epitope regions to be integrated corresponds to a first signal intensity value;
sorting each of the strong signal short peptides by the first signal intensity values corresponding to each of the strong signal short peptides in the set of the epitope regions to be integrated being ranked in descending order;
for each of the sorted strong signal short peptides, performing the following spatial alignment record sorting operation according to the ranking order of the strong signal short peptide:
for each of the spatial alignment records in the set of spatial alignment records corresponding to the strong signal short peptide, sorting each of the spatial alignment records of the short peptide by ranking spatial alignment score of each of the spatial alignment records in descending order;
performing the following existing region generation operation for each of the sorted strong signal short peptides and each of the corresponded sorted spatial alignment records:
taking the amino acid site that aligned with the target antigen in the first-ranking spatial alignment record corresponding to the first-ranking strong signal short peptide as a seed, adding the seed into an existing region, wherein the existing region is an intermediate state region before completing the region growth operation;
based on the existing region, selecting a strong signal short peptide and each of its corresponded spatial alignment records from each of the sorted strong signal short peptides and each of the corresponded sorted spatial alignment records according to the ranking order of each of the strong signal short peptides, and performing the following existing region growth operation:
determining whether the site in the spatial alignment record comprises a site in the existing region, and the number of sites comprised in the existing region is not less than the number of sites not comprised in the existing region;
if yes, adding sites not comprised in the existing region in the spatial alignment record into the existing region;
if no, performing the region growth operation on the next spatial alignment record corresponding to the strong signal short peptide or the next strong signal short peptide by the ranking order of each of the spatial alignment records corresponding to the strong signal short peptide or the ranking order of each of the strong signal short peptides;
if the sites in the existing region increases after iterating over the strong signal short peptides for once, iterating over all the strong signal short peptides again until the sites in the existing region no longer increase, and completing the region growth operation;
for each of the sorted spatial alignment records corresponding to the strong signal short peptide, determining whether at least one spatial alignment record in all spatial alignment records corresponding to the strong signal short peptide comprises a site in a first existing region;
if no, adding the strong signal short peptide and the corresponding ranking order thereof into a set of candidate seeds;
for each of the strong signal short peptides in the set of candidate seeds, performing the existing region generation operation and the existing region growth operation according to the ranking order corresponding to each of the strong signal short peptides;
updating the set of candidate seeds after the existing region growth operation completed until no new seed can be screened;
acquiring all existing regions which have completed the growth, adding the sites in each existing region into a set of spatially alignment regions, and generating the spatially alignment region.

9. The method of claim 1 wherein said identifying key binding sites of the antigenic epitope comprised in the spatially alignment region based on the spatially alignment region comprises:
determining whether the amino acid site is comprised in a key amino acid identifier sequence in any of corresponding spatial alignment records based on amino acid sites aligned with the target antigen in the spatial alignment region;
if yes, the amino acid site is a key binding site of epitope in the targeted protein.

10. A method for generating an amino acid identifier sequence with a specific length, comprising:
generating an amino acid identifier sequence corresponding to an amino acid sequence, performing the following segmentation operation and generating a set of first subsequences: segmenting the amino acid identifier sequence into first subsequences comprising preset numbers of amino acid identifiers, wherein the amino acid identifiers in the first subsequence appear continuously in the amino acid identifier sequence;
for each amino acid identifier sequence in the set of amino acid identifier sequences, performing a space addition operation and generating a set of second subsequences, wherein the second subsequences are amino acid identifier subsequences comprising preset numbers of amino acid identifiers and the amino acid identifiers in the second subsequence appear non-continuously in the amino acid identifier sequence;
adding the set of first subsequences and the set of second subsequences into the set of amino acid identifier subsequences to generate the set of amino acid identifier subsequences;
wherein said performing a space addition and generating a set of second subsequences comprises:
setting that the first and last positions of each amino acid identifier sequence in the set of amino acid identifier sequences are not spaced, determining whether the number of remaining amino acid identifiers in the second subsequence generated after space addition is greater than or equal to a preset space addition number, wherein the number of remaining amino acid identifiers is the number of remaining amino acid identifiers excluding the first and last amino acid identifiers;
if yes, performing a space addition operation on the full amino acid identifier subsequence composed of amino acid identifiers, wherein the space addition operation comprises:
replacing amino acid identifiers in the full amino acid identifier subsequence one by one using from 1 to n designated placeholders to generate a second subsequence with n spaces added;
where n is a preset number of spaces added;
if no, performing an amino acid identifier addition operation on an all-empty subsequence formed by the designated placeholder, wherein the amino acid identifier addition operation comprises:
replacing designated placeholders one by one using from 1 to L−2−n amino acid identifiers to generate a second subsequence comprising L−2−n remaining amino acid identifiers;
wherein L is a preset length of the second subsequence, and n is a preset number of spaces added.
